(19) United States
(12) Patent Application Publication
(10) Pub. No.: US 2004/0229827 A1
(43) Pub. Date: Nov. 18, 2004

(54) Dna immunization vectors

(76) Inventors: Michael Steward, Essex (GB); Vivienne Frances Cox, Kent (GB)

Correspondence Address: HELLER EHRMAN WHITE & MCAULIFFE LLP, 1666 K STREET,NW, SUITE 300, WASHINGTON, DC, 20006, US

(21) Appl. No.: 10/257,047

(22) PCT Filed: Apr. 9, 2001
(86) PCT No.: PCT/GB01/01599

(30) Foreign Application Priority Data
Apr. 8, 2000 (GB) 0008582.9

(51) Int. Cl. A61K 48/00; C07H 21/04; C12N 15/85

(52) U.S. Cl. 514/44; 536/23.1; 435/455

(57) ABSTRACT

The invention describes the construction of murine, human and non-human primate variant DNA sequences encoding proteins, such as C3d units, which can be ligated in tandem with each other with or without the native (wild-type) protein (C3d) DNA sequence and may be stably maintained in prokaryotic and eukaryotic expression vectors to produce concatamers of two, three or four copies of either murine, human or non-human primate protein (C3d) at commercially viable levels, and their use in DNA immunization vectors with reduced capacity for homologous integration into host genomic DNA. The invention describes: the construction of novel synthetic DNA sequences encoding concatamers of murine, human or non-human primate C3d where the polypeptide sequence of each unit of the C3d is identical, but the DNA encoding each unit is unique; high-level expression of concatamers of murine, human or non-human primate C3d in prokaryotic and eukaryotic systems and maintenance of stable recombinant expression vector stocks; and the use of variant C3d genes fused to antigen in a DNA immunization vector. The invention also provides a process for preparing oligomeric polypeptides (proteins) in vitro or in vivo which process comprises construction of a DNA vector encoding said polypeptide and its introduction into a recombinant host cell in vitro or host organism in vivo and providing conditions under which said polypeptide will be expressed. The variant DNA polymer comprising a nucleotide sequence that encodes the polypeptide also forms part of the invention.

This invention relates to novel genetic constructs designed to permit expression of a naturally-occurring polypeptide from non-native variant DNA sequences, which, when used to express concatamers of the polypeptide show enhanced stability, leading to high level expression in eukaryotic and prokaryotic cell expression systems and when incorporated into a DNA immunization vector reduce the risk of such sequences undergoing homologous recombination with genomic DNA, thus reducing the risk of potentially damaging integration events.

Naturally occurring immune modulators, such as cytokines, or as described below, proteins derived from the complement system, can enhance specific immune responses to an antigen. A number of these have been proposed for inclusion into DNA immunization vectors to be expressed concurrently with the antigen (reviewed by Leitner et al., 1999 Vaccine 18: 765-77). The use of naked DNA as an immunogen has raised concerns about the potential for its integration into the human genome and the possibility of insertional mutagenesis resulting in the inactivation of tumor suppressor genes or the activation of oncogenes (reviewed by Nicholls et al., 1995 Ann NY Acad Sci 772: 30-9). Although the studies reviewed by Nicholls et al., (1995) have shown this to be a low frequency occurrence with plasmids containing non-human sequences, the inclusion of genes derived from the human genome increases this risk significantly.

This invention may be used in any context where a nucleic acid sequence is included in a medicament where the sequence of the nucleic acid is homologous to a sequence in the genome of the recipient human or animal host. These may be used in the context of gene therapy, therapeutic or prophylactic vaccination or other therapeutic strategies in which nucleic acid forms part of the medicament. It is particularly useful for, but is not restricted to, DNA immunization vectors encoding proteins with immunopotentiating properties derived from the complement system.

The complement system consists of a set of serum proteins that are important in the response of the immune system to foreign antigens. The complement system becomes activated when its primary components are cleaved and the products, alone or with other proteins, activate additional complement proteins resulting in a proteolytic cascade. Activation of the complement system leads to a variety of responses including increased vascular permeability, chemotaxis of phagocytic cells, activation of inflammatory cells, opsonisation of foreign particles, direct killing of cells and tissue damage. Activation of the complement system may be triggered by antigen-antibody complexes (the classical pathway) or a normal slow activation may be amplified in the presence of cell walls of invading organisms such as bacteria and viruses (the alternative pathway). The complement system interacts with the cellular immune system through a specific pathway involving C3, a protein central to both classical and alternative pathways. The proteolytic activation of C3 gives rise to a large fragment (C3b) and exposes a chemically reactive internal thiolester linkage which can react covalently with external nucleophiles such as the cell surface proteins of invading organisms or foreign cells. As a result, the potential antigen is ‘tagged’ with C3b and remains attached to that protein as it undergoes further proteolysis to iC3b and C3d,g. The latter fragments are, respectively, ligands for the complement receptors CR3 and CR2. Thus the labeling of antigen by C3b can result in a targeting mechanism for cells of the immune system bearing these receptors.

That such targeting is important for augmentation of the immune response is first shown by experiments in which mice were depleted of circulating C3 and then challenged with an antigen (sheep erythrocytes). Removal of C3 reduced the antibody response to this antigen. (M. B. Pepys, J. Exp. Med, 140, 126-145, 1974). The role of C3 was confirmed by studies in animals genetically deficient in either C3 or the upstream components of the complement cascade which generate C3b, i.e. C2 and C4, (J. M. Ahearn & D. T. Fearon, Adv. Immunol. 46, 183-219, 1989). More recently, it has been shown that linear conjugation of a model antigen with more than two copies of the murine C3d fragment sequence resulted in a very large (1000-10000-fold) increase in antibody response in mice compared with unmodified antigen controls (P. W. Dempsey et al, Science, 271: 348-350, 1996; WO96/17625, PCT/GB95/02851). The increase could be produced without the use of conventional adjuvants such as Freund's complete adjuvant. The mechanism of this remarkable effect was demonstrated to be high-affinity binding of the multivalent C3d construct to CR2 on B-cells, followed by co-ligation of CR2 with another B-cell membrane protein, CD19, and with membrane-bound immunoglobulin to generate a signal to the B-cell nucleus.

In the experiments of Dempsey et al, (1996) the unmodified antigen control and linear fusions with one or two C3d domains were prepared by transfection of the appropriate coding plasmids into L cells followed by the selection of high-expressing clones. The most immunogenic construct, that with three C3d units, had to be expressed transiently in COS cells and this procedure gave a very poor yield of the fusion protein. In part, the low yield could be attributed to the generation of species containing the antigen but with lower molecular weights, corresponding to fewer than three C3d units. It was unclear from the published work of Dempsey et al whether the latter molecules originated by proteolysis of the three-C3d construct or whether they were due to a recombination event in vivo.

Using another expression system but the same C3d constructs as Dempsey et al, we obtained evidence that the generation of molecules with <3 C3d units from DNA encoding 3× C3d repeats is due to loss of one or more C3d units by homologous recombination and not due to post-translational processing (see WO99/35260) and described methods for the generation and selection of stable variant genes resistant to homologous recombination.

The present invention describes the construction of murine, human and non-human primate variant DNA sequences encoding C3d units which can be ligated in tandem with each other with or without the native (wild-type) C3d DNA sequence and may be stably maintained in prokaryotic and eukaryotic expression vectors to produce concatamers of two, three or four copies of either murine, human or non-human primate C3d at commercially viable levels, and their use in DNA immunization vectors with reduced capacity for homologous integration into host genomic DNA.

The invention comprises the following elements:

1. The construction of novel synthetic DNA sequences encoding concatamers of murine, human or non-human primate C3d where the polypeptide sequence of each unit of the C3d is identical, but the DNA encoding each unit is unique.

2. High-level expression of concatamers of murine, human or non-human primate C3d in prokaryotic and eukaryotic systems and maintenance of stable recombinant expression vector stocks.

3. The use of variant C3d genes fused to antigen in a DNA immunization vector.

The above steps involve the following general processes:

The invention provides a process for preparing oligomeric polypeptides in vitro or in vivo according to the invention which process comprises construction of a DNA vector encoding said polypeptide and its introduction into a recombinant host cell in vitro or host organism in vivo and providing conditions under which said polypeptide will be expressed. That process may comprise the steps of:

(i) preparing a replicable expression vector comprising a nucleotide sequence that encodes said polypeptide;

ii) transforming a host cell with said vector;

iii) culturing said transformed host cell under conditions permitting replication of said expression vector or to produce said polypeptide; and

iv) recovering said expression vector in a form suitable for DNA immunization or said polypeptide in an active form.

The variant genes' DNA polymer comprising a nucleotide sequence that encodes the polypeptide also forms part of the invention.

The process of the invention may be performed using conventional recombinant techniques such as described in Sambrook et al., Molecular Cloning: A laboratory manual 2nd Edition. Cold Spring Harbor Laboratory Press (1989) and DNA Cloning vols I, II and III (D. M. Glover ed., IRL Press Ltd).

The invention also provides a process for preparing the DNA polymer by the condensation of appropriate mono-, di- or oligomeric nucleotide units.

The preparation may be carried out chemically, enzymatically, or by a combination of the two methods, in vitro or in vivo as appropriate. Thus, the DNA polymer may be prepared by the enzymatic ligation of appropriate DNA fragments, by conventional methods such as those described by D. M. Roberts et al., in Biochemistry 1985, 24, 5090-5098.

The DNA fragments may be obtained by digestion of DNA containing the required sequences of nucleotides with appropriate restriction enzymes, by chemical synthesis, by enzymatic polymerisation, or by a combination of these methods.

Digestion with restriction enzymes may be performed in an appropriate buffer at a temperature of 20′-70° C., generally in a volume of 50 μl or less with 0.1-10 μg DNA. Enzymatic polymerisation of DNA may be carried out in vitro using a DNA polymerase such as DNA polymerase 1 (Klenow fragment) in an appropriate buffer containing the nucleoside triphosphates DATP, dCTP, dGTP and dTTP as required at a temperature of 10°-37° C., generally in a volume of 50 μl or less. Enzymatic ligation of DNA fragments may be carried out using a DNA ligase such as T4 DNA ligase in an appropriate buffer at a temperature of 4° C. to 37° C., generally in a volume of 50 μl or less.

The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes M. Singh, B. S. Sproat and R. C. Titmas, Nucleic Acids Research, 1982, 10, 6243; B. S. Sproat and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus and H. Koester, Nucleic Acids Research, 1984, 12, 4539; and H. W. D. Matthes et al., EMBO Journal, 1984, 3, 801. Preferably an automated DNA synthesiser (for example, Applied Biosystems 381A Synthesiser) is employed.

The DNA polymer is preferably prepared by ligating two or more DNA molecules which together comprise a DNA sequence encoding the polypeptide. The DNA molecules may be obtained by the digestion with suitable restriction enzymes of vectors carrying the required coding sequences.

The precise structure of the DNA molecules and the way in which they are obtained depends upon the structure of the desired product. The DNA molecule encoding the polypeptide may be constructed using a variety of methods including chemical synthesis of DNA oligonucleotides, enzymatic polymerisation, restriction enzyme digestion and ligation. The design of a suitable strategy for the construction of the DNA molecule coding for the polypeptide is a routine matter for the skilled worker in the art.

The expression of the polypeptide encoded by the DNA polymer in a recombinant host cell or in vivo by a recipient of a DNA immunisation vector may be carried out by means of a replicable expression vector capable, in the host cell, of expressing the polypeptide from the DNA polymer.

The replicable expression vector may be prepared in accordance with the invention, by cleaving a vector compatible with the host cell to provide a linear DNA segment having an intact replicon, and combining said linear segment with one or more DNA molecules which, together with said linear segment, encode the polypeptide, under ligating conditions.

The ligation of the linear segment and more than one DNA molecule may be carried out simultaneously or sequentially as desired. Thus, the DNA polymer may be preformed or formed during the construction of the vector, as desired. The choice of vector will be determined in part by the host cell, which may be prokaryotic, such as E. coli, mammalian, such as mouse C127, mouse myeloma, Chinese hamster ovary, or other eukaryotic (fungi e.g. filamentous fungi or unicellular yeast or an insect cell such as Drosophila or Spodoptera). The host cell may also be in a transgenic animal or a human or animal recipient of a DNA immunization vector. Suitable vectors include plasmids, bacteriophages, cosmids and recombinant viruses derived from, for example, baculoviruses, vaccinia, adenovirus and herpesvirus.

The DNA polymer may be assembled into vectors designed for isolation of stable transformed mammalian cell lines expressing the fragment e.g. bovine papillomavirus vectors in mouse C127 cells, or amplified vectors in Chinese hamster ovary cells (DNA Cloning Vol. II D. M. Glover ed. IRL Press 1985; Kaufman, R. J. et al. Molecular and Cellular Biology 5, 1750-1759,1985; Pavlakis G. N. and Hamer, D. H. Proceedings of the National Academy of Sciences (USA) 80, 397-401, 1983; Goeddel, D. V. et al. European Patent Application No. 0093619, 1983).

The preparation of the replicable expression vector may be carried out conventionally with appropriate enzymes for restriction, polymerisation and ligation of the DNA, by procedures described in, for example, Sambrook et al., cited above. Polymerisation and ligation may be performed as described above for the preparation of the DNA polymer. Digestion with restriction enzymes may be performed in an appropriate buffer at a temperature of 20°-70° C., generally in a volume of 50 μl or less with 0.1-10 μg DNA.

The recombinant host cell is prepared, in accordance with the invention, by transforming a host cell with a replicable expression vector of the invention under transforming conditions. Suitable transforming conditions are conventional and are described in, for example, Sambrook et al., cited above, or “DNA Cloning” Vol. 1, D. M. Glover ed., IRL Press Ltd, 1985.

The choice of transforming conditions is determined by the host cell. Thus, a bacterial host such as E. coli, may be treated with a solution of CaCl₂ (Cohen et al., Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol or by electroporation as for example described by Bio-Rad Laboratories, Richmond, Calif., USA, manufacturers of an electroporator. Eukaryotic cells in culture may be transformed by calcium co-precipitation of the vector DNA onto the cells or by using cationic liposomes. DNA immunization vectors may be administered as naked DNA or contained within a viral particle by injection or by other means of delivery including aqueous or non-aqueous formulations via transdermal or mucosal routes.

The invention also extends to a host cell transformed with a variable replicable expression vector of the invention.

Culturing the transformed host cell under conditions permitting expression of the DNA polymer is carried out conventionally, as described in, for example, Sambrook et al., and “DNA Cloning” cited above. Thus, preferably the cell is supplied with nutrient and cultured at a temperature below 45° C.

The protein product may be recovered by conventional methods according to the host cell. Thus, where the host cell is bacterial such as E. coli and the protein is expressed intracellularly, it may be lysed physically, chemically or enzymatically and the protein product isolated from the resulting lysate. Where the host cell is eukaryotic, the product is usually isolated from the nutrient medium. Where the host cell is in a transgenic animal the protein product may be recovered from the natural secretory pathways (e.g. where the protein is secreted in the milk of a female transgenic animal). Where the host cell is in a human or animal recipient of a DNA immunization vector or gene therapy vector protein products are not normally recovered, but may be detected in tissues for the purpose of evaluating the utility of the delivery system.

WO99/35260 describes methods for purification and refolding (where required) of protein products expressed in prokaryotic and eukaryotic systems.

The nucleic acid may contain an additional cysteine codon which will be expressed at the carboxy-terminus of the polypeptide described in this invention. The utility and post-translational modification of the carboxy-terminal cysteine is described in WO99/35260.

The use of insect cells infected with recombinant baculovirus encoding the polypeptide portion is a preferred general method for preparing complex proteins, particularly the polypeptide encoding C3d oligomers of the invention or fusions of the C3d oligomers with an antigen. The use of DNA immunization vectors is an alternative general method for delivery of the polypeptide encoding C3d oligomers fused to antigen in vivo as an immunogen for prophylactic or therapeutic purposes.

### General Methods Used in Examples

(i) DNA Cleavage

Cleavage of DNA by restriction endonucleases was carried out according to the manufacturer's instructions using supplied buffers (New England Biolabs (U.K.) Ltd., Herts. or Promega Ltd., Hants, UK). Double digests were carried out simultaneously if the buffer conditions were suitable for both enzymes. Otherwise double digests were carried out sequentially where the enzyme requiring the lowest salt condition was added first to the digest. Once the digest was complete the salt concentration was altered and the second enzyme added.

(ii) DNA Ligation

Ligations were carried out using T4 DNA ligase purchased from Promega or New England Biolabs as described in Sambrook et al, (1989) Molecular Cloning: A Laboratory Manual 2nd Edition, Cold Spring Harbor Laboratory Press.

(iii) Plasmid Isolation

Plasmids were isolated using Wizard™ Plus Minipreps (Promega) or Qiex mini or midi kits and Qiagen Plasmid Maxi kit (QIAGEN, Surrey) according to the manufacturer's instructions.

(iv) DNA Fragment Isolation

DNA fragments were excised from agarose gels and DNA extracted using the QIAEX gel extraction kit or Qiaquick (QIAGEN, Surrey, UK), or GeneClean, or GeneClean Spin Kit or MERmaid Kit, or MERmaid Spin Kit (Bio 101 Inc, CA. USA) gel extraction kits according to the manufacturer's instructions.

(v) Introduction of DNA into E. coli

Plasmids were transformed into competent E. coli BL21(DE3) or XL1-blue strains (Studier and Moffat, (1986), J. Mol. Biol 0.189:113). The E. coli strains were purchased as a frozen competent cultures from Stratagene (Cambridge, UK).

(vi) DNA Sequencing

The sequences were analysed by a Perkin Elmer ABI Prism 373 DNA Sequencer. This is an electrophoretic technique using 36 cm×0.2 mm 4% acrylamide gels, the fluorescently labeled DNA fragments being detected by a charge coupled device camera according to the manufacturer's instructions.

(vii) Production of Oligonucleotides and Synthetic Genes

Oligonucleotides and synthetic genes were purchased from Cruachem, Glasgow, UK or from Sigma-Genosys, Cambridge, UK.

(viii) Generation of Baculovirus Vectors

Plasmids described in this invention having the prefix pBP (e.g. pBP68-03 described below) are used to generate baculovirus vectors and express the encoded recombinant polypeptides by the following methods (Sections (viii) to (x)).

Purified plasmid DNA was used to generate recombinant baculoviruses using the kit ‘The BacPak Baculovirus Expression System’ according to the manufacturer's protocols (Clontech, CA, USA). The insect cell line Sf9 (ATCC) was grown in IPL-41 medium (Sigma, Dorset, UK) supplemented according to manufacturers recommendations with yeast extract, lipids and pluronic F68 (all from Sigma) and 1% (v/v) foetal calf serum (Gibco, Paisley, UK)—this is termed growth medium. Cells were transfected with the linearised baculovirus DNA (supplied in the kit) and the purified plasmid. Plaque assays (see method below) were carried out on culture supernatants and a series of ten-fold dilutions thereof to allow isolation of single plaques. Plaques were picked using glass Pasteur pipettes and transferred into 0.5 ml aliquots of growth medium. This is the primary seed stock.

(ix) Plaque Assay of Baculoviruses

1×10⁶ Sf9 cells were seeded as monolayer cultures in 30 mm plates and left to attach for at least 30 minutes. The medium was poured off and virus inoculum in 100 μl growth medium was dripped onto the surface of the monolayer. The plates were incubated for 30 minutes at room temperature, occasionally tilting the plates to prevent the monolayer from drying out. The monolayer was overlaid with a mixture of 1 ml growth medium and 3% (w/v) “Seaplaque” agarose (FMC, ME) warmed to 37° C. and gently swirled to mix in the inoculum. Once set a liquid overlay of 1 ml growth medium was applied. The plates were incubated in a humid environment for 3-5 days.

Visualisation of plaques was achieved by addition to the liquid overlay 1 ml phosphate buffered saline (PBS) containing neutral red solution at 0.1% (w/v) from a stock solution of 1% (w/v) (Sigma, Dorset, UK). Plaques were visible as circular regions devoid of stain up to 3 mm in diameter.

(x) Scale-Up of Baculovirus Vectors and Protein Expression

200 μl of the primary seed stock was used to infect 1×10⁶ Sf9 monolayer cell cultures in 30 mm plates. The seed stock was dripped onto the monolayer and incubated for 20 minutes at room temperature, and then overlaid with 1 ml growth medium. The plates were incubated at 27° C. in a humid environment for 3-5 days. The supernatant from these cultures is Passage 1 virus stock. The virus titre was determined by plaque assay and further scale up was achieved by infection of monolayer cultures or suspension cultures at a multiplicity of infection (moi) of 0.1. Virus stocks were passaged a maximum of six times to minimise the emergence of defective virus.

Expression of recombinant proteins was achieved by infection of monolayer or suspension cultures in growth medium with or without foetal calf serum (FCS). Where FCS was omitted cells conditioned to growth in the absence of FCS were used. Virus stocks between passage 1 and 6 were used to infect cultures at a moi of >5 per cell. Typically, infected cultures were harvested 72 hours post infection and recombinant proteins isolated either from the supernatants or the cells.

(xi) Protein Purification

A number of standard chromatographic techniques can be used to isolate the C3d-containing proteins, e.g. such methods as ion-exchange and hydrophobic interaction matrixes chromatography utilising the appropriate buffer systems and gradient to purify the target proteins. The properties of the C3d containing fusion polypeptides will vary depending on the nature of the fusion protein. Examples of methods employed in this invention are described in WO99/35260.

(xii) Sodium Dodecyl Sulphate Polyacrylamide Gel Electrophoresis (SDS-PAGE)

SDS-PAGE was carried out generally using the Novex system (Novex GmbH, Heidleburg) according to the manufacturer's instructions. Pre-packed gels of Tris/glycine a 4-20% acrylamide gradient were usually used. Samples for electrophoresis, including protein molecular weight standards (for example LMW Kit, Pharmacia, Sweden or Novex Mark 12, Novex, Germany) were usually diluted in 1% (w/v) SDS-containing buffer (with or without 5% (v/v) 2-mercaptoethanol), and left at room temperature for 5 to 30 min before application to the gel.

(xiii) Immunoblotting

(a) Dot Blot

Immobilon membranes (Millipore, Middlesex, UK) were activated by immersion in methanol for 20 seconds and then washed in PBS for five minutes. The membrane was placed into a vacuum manifold Dot Blotter (Bio-Rad Laboratories, Watford, UK). Crude extracts from cells or culture supernatants were transferred onto the membrane by applying a vacuum and washed through with PBS. Without allowing the membrane to dry out, the Dot Blotter was dismantled and the membrane removed.

(b) Western Blotting

Samples of cell extracts and purified proteins were separated on SDS-PAGE as described in Section (xii). The Immobilon membrane was prepared for use as in (a) above. The gel and the membrane were assembled in the Semi-Dry Transfer Cell (Trans-Blot SD, Bio-Rad Laboratories) with the Immobilon membrane towards the anode and the SDS-PAGE gel on the cathode side. Between the cathode and the gel were placed 3 sheets of Whatman 3M filter paper cut to the size of the gel pre-soaked in a solution of 192 mM 6-amino-n-caproic acid, 25 mM Tris pH 9.4 containing 10% (v/v) methanol. Between the anode and the membrane were placed two sheets of Whatman 3M filter paper cut to the size of the gel and soaked in 0.3M Tris pH 10.4 containing 10% (v/v) methanol next to the anode and on this was laid a further sheet of Whatman 3M filter paper pre-soaked in 25 mM Tris pH 10.4 containing 10% (v/v) methanol.

The whole-assembled gel assembly was constructed to ensure the exclusion of air pockets. The proteins were transferred from the SDS-PAGE to the Immobilon membrane by passing 200 mA current through the assembly for 30 minutes.

(c) Immunoprobing of Dot Blot and Western Membranes

The membranes were blocked by incubating the membrane for 1 h at room temperature in 50 ml of 10 mM phosphate buffer pH 7.4 containing 150 mM NaCl, 0.02% (w/v) Ficoll 400, 0.02% (w/v) polyvinylpyrolidine and 0.1% (w/v) bovine serum albumin (BSA). The appropriate primary antibody was diluted to its working concentration in antibody diluent, 20 mM sodium phosphate buffer pH 7.4 containing 0.3M NaCl, 0.5% (v/v) Tween-80 and 1.0% (w/v) BSA. The membrane was incubated for 2 h at room temperature in 50 ml of this solution and subsequently washed three times for 2 minutes in washing buffer, 20 mM sodium phosphate pH 7.4 containing 0.3M NaCl and 0.5% (v/v) Tween-80. The membrane was then transferred to 50 ml of antibody diluent buffer containing a suitable dilution of the species specific antibody labelled with the appropriate label, e.g. biotin, horse radish peroxidase (HRP), for the development process chosen and incubated for 2 h at room temperature. The membrane was then washed in washing buffer as described above. Finally, the blot was developed according to the manufacturer's instructions.

The appropriate dilution of antibody for both the primary and secondary antibodies refers to the dilution that minimises unwanted background noise without affecting detection of the chosen antigen using the development system chosen. This dilution is determined empirically for each antibody.

(xiv) Gene Sequences

The sequence of wild-type murine and human C3d are available on public databases under accession number K02782 (mouse) and K02765 (human).

### EXAMPLES

### Example 1

### Construction of pBP66-2-14: A Baculovirus Expression Vector Encoding a First Variant of Murine C3d

pBP66-2-14 is a baculovirus expression vector containing a single copy of murine C3d in which the last 72 amino acid codons contain 41 silent changes resulting in 19% divergence from the wild-type sequence over this region and the 18 amino acid linker region contains 21 further silent changes and two additional amino acids compared to the 16 amino acid linker region used in pBP68-01 (described in WO99/35260). The divergence between the linker in pBP66-2-14 and that in pBP68-01 in the region of silent changes is 59%. The silent changes in the sequence may be third base changes such as substitution of GGG for GGC to encode glycine, or may be two or three base substitutions such as the substitution of AGC for TCC or TCT to encode serine. The gene encoding murine C3d within pBP66-2-14 represents a first variant of murine C3d, (SEQ ID 1) which is designed to be expressed as a dimer or trimer with further variants of murine C3d containing different silent changes. pBP66-2-14 was constructed in five steps as described below.

i) Construction of pBP66-05

The vector pBP66-05 was constructed from pBP66-01 (Described in WO99/35260) using site-directed mutagenesis to introduce a site for HindIII at position 2218 without changing the amino acid sequence. The purpose of this change was to allow direct cloning of a variant gene fragment encoding the carboxy-terminal portion of the murine C3d gene from position 2218 to position 2303. Mutagenesis upon pBP66-01 with the oligonucleotides SEQ ID 2 and SEQ ID 3 and transformation of E. coli XL1-blue cells were carried out using the QuikChange Kit (Stratagene) according to the manufacturers instructions. The clone pBP66-05 was selected from transformants by restriction digest analysis of plasmid DNA with the enzyme HindIII.

ii) Construction of pBP66-54-3

The vector pBP66-54-3 was constructed from pBP66-05 using site-directed mutagenesis to introduce multiple silent changes between the positions 2065 and 2218 without changing the amino acid sequence, thus introducing a “fuzzy” gene patch into the wild-type sequence. Four oligonucleotides, Fuz9, Fuz10, Fuz17 and Fuz22 were used to generate a PCR product as described in Example 3a of WO99/35260. The resultant PCR product would have contained a mixed population of “fuzzy” sequences all encoding the same amino acid sequence (apart from those in which PCR errors had arisen). The PCR product was then used to mutagenise pBP66-05 using the QuikChange Kit (Stratagene) according to the manufacturer's instructions with one variation, whereby said PCR product was used in place of mutageneic oligonucletides at a final concentration typically in the range 1 to 100 ng/ml. The clone pBP66-54-3 was selected from transformants by restriction digest analysis of plasmid DNA with the enzyme FokI and the integrity of the sequence was confirmed by DNA sequencing.

iii) Construction of pBS-MF2

pBS-MF2 is a holding vector containing the carboxy terminal region of murine C3d cloned from a “fuzzy” PCR product. The fuzzy PCR fragment was derived from four oligonucleotides Fuz11, Fuz12, Fuz21 and Fuz24 as described in WO99/35260. Fuz11 and Fuz12 were overlapping oligonucleotides encoding the carboxy terminal region of murine C3d, plus the linker peptide (Ser-Gly-Gly-Gly-Gly)₂. Fuz21 and Fuz24 were used to amplify the product of Fuz11 and Fuz12. The four oligonucleotides were used to generate a PCR product as described in example 3a of WO99/35260. The resultant PCR product contained a mixed population of “fuzzy” sequences all encoding the same amino acid sequence (apart from those in which PCR errors had arisen). The PCR product was digested with the enzymes HindIII and EagI, and the 124 bp fragment was purified by agarose gel electrophoresis and ligated with the large fragment (2912 bp) of pBlueScript II KS+ (Stratagene Europe, The Netherlands) digested with the same enzymes and purified in the same way. The ligated DNAs were transformed into E coli XL1 blue and resulting transformants were analysed for the insert by PCR screening using oligonucleotides SED ID 4 and SEQ ID 5. The integrity of the sequence was confirmed by DNA sequencing and pBS-MF2 was selected for the next stage of fuzzy murine C3d construction.

iv) Construction of pBP66-54-3/MF#2

The plasmid pBP66-54-3 was subjected to restriction enzyme digestion with the enzymes HindIII and EagI. Two fragments were generated, one of 280 bp and one of 450 bp. The 450 bp fragment was purified by agarose gel electrophoresis.

The plasmid pBS-MF#2 was subjected to restriction enzyme digestion with the enzymes HindIII and EagI. The 175 bp fragment generated was purified by agarose gel electrophoresis and put into ligation reaction with approximately equimolar amount of the 450 bp fragment from pBP66-54-3. The ligation reaction was then used as template for a PCR using oligonucleotides SEQ ID 6 and SEQ ID 7.

The primers were designed to amplify up the 625 bp product of the ligation, introducing a HindIII site at either terminus. The product of the PCR was gel extracted and ligated into the T-vector pCR2.1 (InVitrogen), the sequence determined and the insert then excised with HindIII. This fragment was introduced into the original 54.3 vector which had been digested with HindIII, thus introducing the variant F region onto the end of the variant A-E region to create the plasmid pBP66-54-3/MF#2.

v) Mutagenesis of pBP66-54-3/MF#2 to Create pBP66-2-14

Errors, probably arising through PCR, were identified in pBP66-54-3/MF#2 Where repairs were required, oligonucleotides were designed spanning a region of 52 bases around the error. Additional changes were also included in the repair oligonucleotides in order to introduce further silent changes to increase divergence from the sequence of pBP66-01. One change resulted in the introduction of a BsrI restriction site which was used for diagnostic purposes following mutagenesis. The mutagenic oligonucleotides are shown in SEQ ID 8 and SEQ ID 9 and mutagenesis was carried out using the QuikChange Kit (Stratagene) according to the manufacturers instructions. In addition, a Kpn 1 site was required after the murine C3d coding and the linker region to facilitate subsequent cloning. This was introduced into pBP66-54-3/MF2 by site-directed mutagenesis. The mutagenic oligonucleotides are shown in SEQ ID 10 and 11 and mutagenesis was carried out using the QuikChange Kit (Stratagene) according to the manufacturer's instructions. After the mutagenesis reactions the resulting transformants were subjected to screening by KpnI or BsrI restriction digest analysis of either plasmid DNA or a PCR product spanning the sites of mutagenesis. The integrity of the sequence was conformed by DNA sequencing.

### Example 2

### Construction of pBP66-26-15: A Baculovirus Expression Vector Encoding a Second Variant of Murine C3d

pBP66-26-15 is a baculovirus expression vector containing a single copy of murine C3d in which the first 219 amino acid codons contain 135 silent changes resulting in 20.5% divergence from the wild-type sequence over this region, plus two additional silent changes in the remaining sequence. All the silent changes in pBP66-26-15 were within codons not altered in the corresponding sequence in pBP66-2-14. The silent changes in the sequence were mostly third base changes such as substitution of GGG for GGC to encode glycine, but also included two or three base substitutions such as the substitution of AGC for TCC or TCT to encode serine. This represents a second variant of murine C3d (SEQ ID 12), which is designed to be expressed as a dimer or trimer with further variants of murine C3d containing different silent changes. The sequence divergence between the 296 amino acid homologous regions of the first and second variants of murine C3d is 20%.

The vector pBP66-26-15 was constructed from pBP66-01 using site-directed mutagenesis to introduce multiple silent changes between the amino acids Thr(T)₁ and Asn(D)₂₁₉ of the murine C3d sequence without changing the amino acid sequence, thus introducing “fuzzy” gene patches into the wild-type sequence. Sixteen oligonucleotides, in four groups of four were used to generate four PCR products as described in example 3a of WO99/35260. Group A contained Fuz1, Fuz2, Fuz23 and Fuz20, Group B contained Fuz3, Fuz4, Fuz19 and Fuz14, Group C contained Fuz5, Fuz6, Fuz13 and Fuz15 and Group D contained Fuz7, Fuz8, Fuz16 and Fuz18. Each of the resultant PCR products A to D would have contained a mixed population of “fuzzy” sequences all encoding the same amino acid sequence (apart from those in which PCR errors had arisen). The PCR products A to D were then used sequentially to mutagenise pBP66-01 using the QuikChange Kit (Stratagene) according to the manufacturer's instructions with one variation, whereby said PCR products were used in place of mutageneic oligonucletides at a final concentration typically in the range 1 to 100 ng/ml.

After each mutagenesis reaction the resulting transformants were subjected to screening by restriction digest analysis of either plasmid DNA or a PCR product spanning the site of muatgenesis, where introduction of “fuzzy” sequence was presumed to remove restriction sites present in the sequence of pBP66-01. The integrity of the sequence was confirmed by DNA sequencing. One or more clones was selected at each stage for subsequent mutagenesis by another of the four PCR products, or repair of PCR-generated errors where no clones with correct sequence could be identified.

Where repairs were required, oligonucleotides were designed spanning a region of 20-60 bases around the error. Additional changes were also included in such repair oligonucleotides in order to introduce further silent changes to increase divergence from the sequence of pBP66-01 or to introduce or remove restriction sites for diagnostic or cloning purposes. Mutagenesis was carried out using the QuikChange Kit (Stratagene) according to the manufacturer's instructions. After each mutagenesis reaction the resulting transformants were subjected to screening by restriction digest analysis of either plasmid DNA or a PCR product spanning the site of muatgenesis using restriction enzymes corresponding to the diagnostic sites generated by the mutagenesis. The integrity of the sequence was confirmed by DNA sequencing.

When the murine C3d sequence encoded by pBP66-01 had been subjected to silent mutagenesis by all four PCR products A to D, and repaired as required to correct PCR-generated errors, one further mutagenesis reaction was carried out. At the start of the coding sequence it was necessary to alter the reading frame such that the signal peptide and the coding sequence would be in the same frame. A Kpn 1 site was also introduced to facilitate subsequent cloning. The mutagenic oligonucleotides are shown in SEQ ID 13 and SEQ ID 14 and mutagenesis was carried out using the QuikChange Kit (Stratagene) according to the manufacturers instructions. After each mutagenesis reaction the resulting transformants were subjected to screening by KpnI restriction digest analysis of either plasmid DNA or a PCR product spanning the site of mutagenesis. The integrity of the sequence was confirmed by DNA sequencing.

### Example 3

### Construction of pBP67-03 Containing a Third Variant of Murine C3d

pBP67-03 is a baculovirus expression vector containing a two copies of murine C3d, the first of which is a third variant of murine C3d (SEQ ID 15) containing 347 changes relative to the wild-type sequence and the second copy is the wild-type murine C3d sequence. The third variant of murine C3d was designed and synthesised de novo with the maximum variation at the DNA level from the first and second variants of murine C3d described in Examples 1 and 2, but encoding an identical polypeptide. The sequence was designed according to the principles of codon variation described in WO99/35260, which takes into account the avoidance of rare codons and was synthesised by Sigma-Genosys (UK), where it was also cloned into the vector pBP66-01 at a unique BglII site to provide in frame fusion of the two murine C3d fragments and allow expression of a concatameric polypeptde encoding two murine C3d units.

### Example 4

### Construction pCR-Yellow Containing a Fourth Variant of Murine C3d

The first and second variants of murine C3d contain regions of the DNA sequence which are identical to the wild-type sequence. A fourth variant of murine C3d was constructed as a fusion of approximately one third of the sequence from the first variant with approximately two thirds of the sequence from the second variant to generate a sequence containing all the silent changes introduced into both variants. This was achieved by PCR amplification of the variable region from the plasmids pBP66-2-14 containing the first variant and pBP66-26-15 containing the second variant. The oligonucleotide primers used for PCR amplification of pBP66-2-14 are given in SEQ ID 16 and SEQ ID 17, and for PCR amplification of pBB66-26-15 are given in SEQ ID 18 and SEQ ID 19.

The two PCR products were digested with the restriction enzymes BseRI, which cleaves between the wild-type and variant sequence in both PCR products and were purified by gel electrophoresis. The digested fragments containing the variant sequences were ligated in vitro. A PCR reaction was carried out to amplify the full-length variant murine C3d sequence using the oligonucleotide primers SEQ ID 16 and SEQ ID 19, and the PCR product was cloned into the vector pCR2.1 (InVitrogen) by T-cloning according to manufacturer's instructions, and the sequence of the resultant plasmid pCR-yellow was authenticated by sequence analysis. The coding sequence of the fourth variant of murine C3d is given in SEQ ID 20.

### Example 5

### Ligation of Three Variants of Murine C3d in a Single Concatamer

The fourth variant of murine C3d was excised from pCR-yellow using the restriction enzymes BglII and BamHI. The 960 base-pair fragment was purified by gel electrophoresis and cloned into the unique BglII site of pBP67-03, which encodes a concatamer of the third (synthetic) variant of murine C3d and wild type murine C3d. The correct orientation of the fourth variant was determined by PCR screening. The resulting plasmid, pBP68-03 is a baculovirus transfer vector containing three copies of murine C3d expressible as a concatamer, where the sequence of each copy differs by 20-35%. The sequence of the region of pBP68-03 encoding the murine C3d concatamer and its signal peptide is given in SEQ ID 21.

### Example 6

### Expression of Stable Murine C3d Oligomers in Insect Cells Using pBP68-03

Expression of murine C3d oligomers using duplicated wild-type sequence in insect cells using the baculovirus expression system was described in WO99/35260, where it was observed that three copies of murine C3d generated a product corresponding to only a single C3d unit, and that the loss of the other two units was due to homologous recombination at the DNA level resulting in deletion of two of the identical DNA sequences encoding the murine C3d units. In this example the plasmids pBP67-03 and pBP68-03 were used to produce recombinant baculoviruses using the methods described above. High levels of murine C3d dimer (including a carboxy-terminal cysteine) were produced by baculoviruses derived from pBP67-03, and of murine C3d trimer by baculoviruses derived from pBP68-03 and the production of the intact oligomeric product was stable over multiple passages of the recombinant baculovirus stock permitting scale-up to large volumes and commercially viable amounts of protein (5-30 mg/litre of culture).

### Example 7

### Construction of DNA Immunization Vectors Using Variant Murine C3d Sequences

A model system for DNA immunization in humans is the mouse, where immune responses to antigens produced may be monitored. These models may also be used to determine the frequency of genomic integration events using methods described by Nicholls et al., (1995 Ann NY Acad Sci 772: 30-9) and the safety profile of such vectors may be evaluated. DNA encoding two of the murine C3d variants with a single copy of wild-type murine C3d were cloned into the DNA immunization vector pVAX1 (Invitrogen) in tandem with DNA encoding the antigen Plasmodium yoelii MSP1.19.

a) Construction of pVAX3: A DNA Immunization Vector for Efficient In Vivo Expression of Recombinant Proteins.

The vector pVAX1 was modified prior to insertion of the murine C3d sequences. The multiple cloning site was removed by digestion with PmeI restriction enzyme and replaced with a synthetic oligonucleotide linker containing the signal peptide sequence from human tissue plasminogen activator (tPA) to create pVAX2. The linker also included sites for BglII and BamHI restriction enzymes, followed by two stop codons. The sequence of the inserted DNA is given in SEQ ID 22 and 23

The pVAX2 vector was subsequently modified by site-directed mutagenesis to generate the “Kozak” consensus sequence (Kozak, M. 1981 Nucleic Acids Res. 9, 5233-62) at the initiation codon of the tPA leader peptide to make the vector pVAX3. The sequence at this point therefore now reads GCCACCATGG.

b) Construction of pVK68-01: A DNA Immunization Vector Encoding Murine C3d₃

Murine C3d₃ gene cassettes were introduced into the pVAX3 vector by digestion of the vector with BglII and BamHI. The murine C3d₃ cassette was removed from the baculovirus expression vector pBAC68-04 by digestion with the same enzymes and ligated into the pVAX3 DNA to generate the vector pVK68-01. (pBAC68-04 contains the same C3d₃ cassette as pBP68-03 described above, but the holding vector was pBAC1 (Novagen) instead of pBacPak (Clontech)). Correctly assembled clones of pVK68-01 in pVAX3 were identified by the retention of both BglII and BamHI sites, which could then be used for the insertion of genes encoding antigen. The sequence of pVK68-01 is given in SEQ ID 24

c) Construction of pVK96-01 and pVK96-02. DNA Immunization Vectors Encoding a Malaria Antigen Fused at the Amino or Carboxy Terminal of Murine C3d₃.

A synthetic gene encoding the carboxy-terminal fragment of the Plasmodium yoelii MSP1 gene (hereafter described as PyMSP1.19) was synthesised using seven overlapping oligonucleotides, the sequence of which is given in SEQ ID 25 to SEQ ID 31. The amino acid codons within the DNA sequence of PyMSP1.19 were optimized for mammalian expression. The native sequence contains many “rare” codons which were eliminated without affecting the sequence of the encoded polypeptide. The seven oligonucleotides were annealed in vitro and then subjected to a two-step PCR amplification using the following method:

1 pmol of each oligonucleotide M1-M7 was incubated with 200 uM dNTPs, 0.5×Taq ligase buffer, 0.5×Pfu turbo buffer, 5 U Taq ligase and 5 U Pfu turbo polymerase in a total volume of 50 ul. The reaction was subjected to 15 cycles of PCR. The PCR product was then further amplified by the addition of 25 pmol of two primers designed to the termini of the synthetic to 5 ul of the first step PCR reaction, 200 uM dNTPs and 1×Pfu turbo polymerase in a total volume 50 ul for a further 35 PCR cycles.

The reaction product was gel extracted, and 5 ul were incubated with Taq DNA polymerase for 30 minutes at 72° C. in the presence of 1× buffer, MgCl₂ (2 mM) and dNTPs (200 uM). This promoted the addition of nontemplated A residues to the 3′ termini, allowing T-cloning of the products. The product was cloned into the holding vector pUC57/T (MBI Fermentas) and the correct sequence was determined by DNA sequencing. The PyMSP1.19 gene was excised from the holding vector with BglII and BamHI and inserted into either the BglII site or BamHI site of pVK80-01 to fuse the antigen sequence in frame at either the amino or carboxy termini of the murine C3d₃ cassette. This generated pVK96-01 (amino-terminal fusion) and pVK96-02 (carboxy-terminal fusion) respectively. The sequence of the coding regions for pVK96-01 and pVK96-02 is given in SEQ ID 32 and 33.

d) Analysis of Protein Products Expressed In Vitro from DNA Immunization Vectors

Expression of the protein product from the vectors was assessed by carrying out transient transfection of COS7 cells. Using a transfection reagent such as Effectene (Qiagen), the plasmid DNA was introduced into a 90% confluent monolayer of COS7 cells, and incubation of the cells continued for a further 72 hours. Samples of the supernatant and cell pellet were analysed by Western blot for the presence of C3d or antigen-containing protein. All DNA immunization vectors derived by insertion of murine C3d₃ with or without PyMSP1.19 into pVAX3 produced immunoreactive material of the expected molecular weight which was secreted into the supernatant. Efficient expression and secretion from cultured cells in vitro provides a degree of confidence that the recombinant fusion proteins will be expressed in vivo after the vectors have been administered to mice as described in Example 8.

### Example 8

### Immunization of Mice with DNA Immunization Vectors Encoding Murine C3d Fused to Antigens

The recombinant DNA immunization vector encoding the murine C3d oligomer-antigen fusions are used to immunize mice using the following protocol. Immunizations are performed using the BioRAD Helios Gene Gun. The plasmid DNA is precipitated onto gold microcarriers in the presence of spermidine, and the gold coated onto the inside of “gold-coat” tubing. The 12.7 mm [0.5″] lengths of tubing are stored desiccated at 4° C. until required for use. A single sample of gold-DNA complex is delivered to the shaved abdomen of mice at 2.758 Mpa [400 psi] of helium pressure. A second immunization was performed six weeks after the initial boost.

Vectors encoding more than a single copy of C3d are demonstrated to have enhanced humoral immune responses to the antigen encoded as a fusion to the C3d concatamer. Vectors in which the C3d sequences are non-identical to wild type C3d show a reduced frequency of integration into the genome in comparison with vectors containing wild-type murine C3d.

It can be inferred from these observations that i) a DNA immunization vector encoding greater than one copy of C3d attached to an antigen shows enhanced humoral immune responses to that antigen, and ii) DNA immunization vectors encoding murine proteins are less likely to integrate into the genome of the host if the DNA encoding the murine protein is non-identical to the gene found within the genome of the host.

### Example 9

### Cloning of Wild-Type Human C3d from a Liver Library and its Expression in E. coli

Human liver total RNA was obtained from Origene Inc. 2 micrograms were used in a conventional reverse transcriptase (RT) reaction with 20 pmol oligonucleotide primer SEQ ID 34. The RT reaction was then subjected to 35 cycles of PCR by appropriate adjustment of buffer conditions and addition of the oligonucleotide primer SEQ ID 35. The generation of an amplicon of 892 bp as visualized on agarose gel electrophoresis was evidence of successful amplification of the human C3d mRNA. The PCR product was cloned directly into the vector pCR2.1 (InVitrogen) by T-cloning according to manufacturer's instructions, and the sequence of pCR-hC3d-2 authenticated by sequence analysis.

This DNA construct was used as template for a subsequent round of PCR amplification to generate a DNA which possessed additional features to facilitate its subcloning into the E. coli expression vector pET26b (Novagen). The following features were introduced at the 5′ end of the gene:

i) The addition of two G bases at the 5 end of the primer to increase the efficiency of T cloning (GG).

ii) The incorporation of an NdeI site incorporating a synthetic initiation ATG codon. (CATATG).

iii) The alteration of the CCC codon for Pro)₃ to CCG (where ATG is codon 1). CC is a rare codon in E coli and its presence at the start of the gene may have compromised expression levels.

iv) The alteration of codon 6 from TGC (Cys) to TCC (Ser) to remove the natural site for thioester linkage of C3d to antigenic surfaces.

Features i) to iv) were introduced with the oligonucleotide primer SEQ ID 36.

The following alterations were made at the 3′ end of the sequence:

v) The addition of two G bases at the 5 end of the primer to increase the efficiency of T cloning (GG).

vi) The addition of codons for five extra amino acids (SSGSC) and a termination codon (TCAGCAGGATCCACTGCT)

vii) The incorporation of an EcoRI restriction enzyme site (GAATTC)

The alterations v) to vii) were made using the oligonucleotide primer SEQ ID 37.

The product of a standard PCR reaction performed using these primers and pCR-hC3d-2 as template was subcloned into the pCR2.1 vector and a selected clone sequenced. The sequence of the human C3d clone hereafter referred to as the “wild-type” sequence is given in SEQ ID 38.

The insert from pCR78-01 was excised using digestion with Nde1 and EcoR1 at the sites incorporated into the PCR primers, gel purified and ligated into pET26b, which had been prepared by digestion with the same restriction enzymes within its multiple cloning site. The two DNAs were ligated together and a recombinant clone selected (pET78-01). The clone was verified by restriction mapping prior to transformation into an appropriate expression strain such as BL21(DE3).

Colonies from the transformation of pET78-01 into the expression strain were grown in LB medium until mid-log phase and the expression of the recombinant protein induced by the addition of IPTG to a final concentration of 1 mM, followed by a further three hours growth. At the end of the growth period, the cells were harvested and a proportion lysed in reducing NuPAGE sample buffer (Novex) and the proteins analysed by SDS-PAGE on a 10% NuPAGE gel. In addition, the nature of the protein was further examined by the use of a polyclonal antiserum against human C3d (The Binding Site Ltd) by Western blotting, which showed the production of an immunoreactive species at the expected molecular weight.

### Example 10

### Synthesis of Variant Human C3d Genes

Two variants of human C3d were designed and synthesised de novo with the maximum variation at the DNA level from each other and from the wild-type human C3d described in Example 9 but encoding an identical polypeptide. The sequence was designed according to the principles of codon variation described in WO99/35260, which takes into account the avoidance of rare codons. The two genes were synthesised by Sigma-Genosys (Cambridge, UK) as a single concatamer, which was cloned into the holding vector pUC18 to generate the vector pUC78-10. The sequence of the two variants is given in SEQ ID 39 and SEQ ID 40.

### Example 11

### Ligation of Two Variants of C3d and One Wild-Type C3d in a Contiguous Concatamer in pBP80-02

a) Construction of pBP78-01, a Baculovirus Transfer Vector Encoding a Single Wild-Type C3d Sequence.

The plasmid pET78-01 was digested with BglII and BamHI to excise the wild-type human C3d sequence. The vector pBP68-01 containing the wild-type murine C3d sequence fused to a signal peptide was digested with the same enzymes to remove the murine sequence. The 960 base-pair fragment from pET78-01 and the 5.5 kilobase-pair band from pBP68-01 were purified by gel electrophoresis and ligated to produce pBP78-01, which contains wild-type human C3d. The correct orientation of the insert was determined by PCR screening and the sequence of each junction was determined by DNA sequencing to ensure that a correct in frame fusion of the signal peptide and human C3d had occurred.

b) Construction of pBP80-01, a Baculovirus Transfer Vector Encoding Three Non-Identical, Concatameric Human C3d sequences.

The two synthetic variants of human C3d were excised from pUC78-01 using the restriction enzymes BglII and BamHI. The 1920 base-pair fragment was purified by gel electrophoresis and cloned into the unique BglII site of pBP78-01, which encodes the wild type human C3d. The correct orientation of the two variants was determined by PCR screening. The resulting plasmid, pBP80-01 is a baculovirus transfer vector containing three copies of human C3d^{•}expressible as a concatamer, where the sequence of each copy differs by approximately 30%. The sequence of the region of pBP80-01 encoding the human C3d concatamer and the signal peptide is given in SEQ ID 41.

### Example 12

### Expression of Stable Human C3d Monomer and Oligomers in Insect Cells Using pBP78-01 and pBP80-01

The plasmids pBP78-01 and pBP80-01 were used to produce recombinant baculoviruses using the methods described above. High levels of human C3d monomer (including a carboxy-terminal cysteine) were produced by baculoviruses derived from pBP78-01, and of human C3d trimer by baculoviruses derived from pBP80-01 and the production of the intact oligomeric product was stable over multiple passages of the recombinant baculovirus stock permitting scale-up to large volumes and commercially viable amounts of protein (50-100 mg/litre of culture).

### Example 13

### Construction of a DNA Immunization Vectors Using Variant Human C3d Sequences

a) Construction of pVAX80-01: A DNA Immunization Vector Encoding Human C3d₃.

Human C3d₃ gene cassettes were introduced into the pVAX3 vector by digestion of the vector with BglII and BamHI. The inserts were removed from pBP80-01 (the baculovirus expression vector for human C3d₃) and by digestion with the same enzymes and ligated into the pVAX3 DNA. Correctly assembled clones for human C3d₃ (pVK80-01) in pVAX3 were identified by the retention of both BglII and BamHI sites, which could then be used for the insertion of genes encoding antigen. The sequence of the coding region from pVK80-01 is given in SEQ ID 42.

b) Construction of pVK104-01 and pVK104-02. DNA Immunization Vectors Encoding a Malaria Antigen Fused at the Amino or Carboxy Terminal of Human C3d₃.

The gene for the carboxy terminal fragment of the MSP1 gene from Plasmodium falciparum (hereafter described as PfMSP1.19) was obtained as a gift from Dr Anthony Holder, National Institute for Medical Research (London). PfMSP1.19 was PCR amplified and subcloned into a holding vector using primers which introduced a BglII site at the amino terminus and a BamHI site and a Gly-Gly-Gly-Ser-Gly spacer at the carboxy terminus. The sequence of the PfMSP1.19 insert in pUC105-01 is given in SEQ ID 43. It was excised from pUC105-01 with BglII and BamHI and inserted into either the BglII site or BamHI site of pVK80-01 to fuse the antigen sequence in frame at either the amino or carboxy termini of the human C3d₃ cassette. This generated pVK104-01 (amino-terminal fusion) and pVK104-02 (carboxy-terminal fusion) respectively. The sequence of the coding region from pVK104-01 and pVK104-02 are given in SEQ ID 44 and SEQ ID 45.

c) Construction of pVK104-03 and pVK104-04. DNA Immunization Vectors Encoding a Variant Malaria Antigen Fused at the Amino or Carboxy Terminal of Human Cd3₃.

A variant of PfMSP1.19 was designed such that two cysteine residues that normally form a disulphide bond were converted to other residues. The aim of these mutations was to create an immunogen better able to elicit a protective response than the native amino acid sequence. The rationale for this approach has been described in WO 00/63245. To achieve this the clone pUC105-01 was subjected to site-directed mutagenesis to generate pUC105-03 in two steps, the first to convert Cysteine 12 to Isoleucine, then Cysteine 28 to Tryptophan. The sequence of the altered PfMSP1.19 mutant is given in SEQ ID 46. It was excised from pUC105-03 with BglII and BamHI and inserted into either the BglII site or BamHI site of pVK80-01 to fuse the antigen sequence in frame at either the amino or carboxy termini of the human C3d₃ cassette. This generated pVK104-03 (amino-terminal fusion) and pVK104-04 (carboxy-terminal fusion) respectively. The sequence of the coding region from pVK104-03 and pVK104-04 are given in SEQ ID 47 and SEQ ID 48.

d) Analysis of Protein Products Expressed In Vitro from DNA Immunization Vectors

Expression of the protein product from the vectors was assessed by carrying out transient transfection of COS7 cells. Using a transfection reagent such as Effectene (Qiagen), the plasmid DNA was introduced into a 90% confluent monolayer of COS7 cells, and incubation of the cells continued for a further 72 hours. Samples of the supernatant and cell pellet were analysed by Western blot for the presence of C3d or antigen-containing protein. All DNA immunization vectors derived by insertion of human C3d₃ with or without the native or mutant PfMSP1.19 into pVAX3 produced immunoreactive material of the expected molecular weight which was secreted into the supernatant. Efficient expression and secretion from cultured cells in vitro provides a degree of confidence that the recombinant fusion proteins will be expressed in vivo after the vectors have been administered as a vaccine to the recipient as described in example 15.

### Example 14

### Construction of a Third Variant of Human C3d

The vectors PVK80-01, pVK104-01, pVK104-02, pVK104-03 and pVK104-04 contain two variant human C3d genes and one wild-type copy. In order to minimise the risk of integration into the host genome the wild-type gene is replaced with a third variant gene. The third variant is synthesised from overlapping oligonucleotides which, when annealed and amplified produce a synthetic gene with the sequence given in SEQ ID 49. The third variant may also be cloned in tandem with existing sequences encoding three copies of human C3d to generate recombinant proteins with four copies of C3d, either using an expression system such as the baculovirus expression system or in the context of a DNA immunization vector to make the proteins in vivo.

### Example 15

### Immunization of Human and Non-Human Primates with DNA Immunization Vectors Encoding Human or Primate C3d Fused to Antigens

The recombinant DNA immunization vectors encoding the human C3d oligomer-antigen fusions are used to immunize humans or non-human primates. The DNA immunization vectors are delivered by a method suitable for delivery of DNA in a clinical protocol, for example, but not restricted to, intramuscular injection. The pVAX1 vector used in the construction of the DNA immunization vectors described above is suitable for human use and conforms to current FDA guidelines for DNA immunization vectors.

From the studies described in Example 7 it may be inferred that I) a human DNA immunization vectors encoding greater than one copy of human C3d attached to an antigen show enhance humoral immune responses to that antigen, and ii) DNA immunization vectors encoding human proteins are less likely to integrate into the genome of the host if the DNA encoding the human protein is non-identical to the gene found within the genome of the host.

Where it is required for the DNA immunization vectors to be tested in a non-human primate species such as rhesus macaques it may be preferred to use C3d sequences which are exactly matched to the species to be used, in order to minimise immune responses to the C3d component of the encoded protein. The following example (16) describes the cloning of wild-type C3d from a rhesus macaques and the design and synthesis of three species-matched variant genes to use in primate models of human disease to test the safety and immunogenicity of the equivalent DNA immunization vectors encoding human C3d.

### Example 16

### Cloning of C3d from Rhesus Liver Tissue Using Degenerate Primers

The sequence of the wild-type C3d sequence from rhesus macaque was obtained by cloning the native sequence from liver using the following method:

a) Primer Design.

The degenerate primers used to clone the Rhesus macaque-specific C3d sequences were designed by alignment of existing C3 protein sequences from human, mouse, rat, and guinea pig. Regions of amino acid conservation within and flanking the C3d region, where low codon redundancy was prominent were selected by eye, and oligonucleotides for RT-PCR designed to incorporate redundant bases where necessary. The primers, which may be used to clone any mammalian C3d sequence, were designated FARM 1 to FARM 8. The sequence of FARM 1-8 is given in SEQ ID 50 to SEQ ID 57.

b) Reverse Transcription-PCR

Total RNA was purified from rhesus macaque liver by the acid-guanidinium thiocyanate-phenol chloroform extraction technique of Chomczynski and Sacchi (Anal. Biochem 162: 156-159 (1987)). Approximately 3 ug of RNA was used in the RT reaction using the reverse transcription system from Promega. Reverse transcription was primed with 40 pmol of anti-mRNA sense primer, (i.e. any of the even-numbered primers).

Nested PCR was used to amplify the rhesus macaque C3d in two halves. An outer PCR with primers FARM 4 and 1 was followed by inner PCR with primers FARM 8 and 3 and in a second reaction an outer PCR with primers FARM 5 and FARM 2 were followed by an inner PCR with primers FARM 3 and 8, thus covering the entire C3d region. PCR conditions were typically 95° C. 30 sec, 54° C. 30 sec, 72° C. 60 sec, ×35 cycles.

c) Subcloning and Sequencing of Novel C3d Clones from Rhesus Macaque.

The PCR products derived from rhesus macaque liver were subcloned into pUC57/T (MBI Fermentas) and a minimum of three clones covering any region of C3d were fully sequenced on both strands. Sequence contigs were assembled and aligned using the SeqMan module of the DNAStar software package. The amino acid sequence of wild-type rhesus macaque C3d is given in SEQ ID 58.

### Example 17

### Construction of a Three Variants of Rhesus Macaque C3d

The first, second and third variant of rhesus macaque C3d are synthesised from overlapping oligonucleotides which, when annealed and amplified produce three synthetic genes with the sequence given in SEQ ID 59 (first variant), SEQ ID 60 (second variant) and SEQ ID 61 (third variant). As in example 9, the cysteine at position 5 is altered to serine in all the variants to prevent aberrant inter- and intra-molecular disulphide formation. These are used to construct DNA immunization vectors containing variant genes encoding species-matched C3d which have a greatly reduced risk of integration into the host genome over vectors containing non-variant genes encoding species-matched sequences. Such DNA immunization vectors encoding rhesus macaque C3d₃ are fused to antigens which have been selected to produce immune responses for the study of human diseases such as malaria, HIV, hepatitis B virus.

1-26. Canceled

27. A DNA immunization vector comprising a linear concatamer of variant DNA sequences encoding C3d which, by virtue of third base redundancy and other variations permissible within an amino acid codon, is non-identical to the naturally occurring DNA sequence encoding C3d.

28. The DNA immunization vector according to claim 27, wherein the variant DNA sequences encodes murine, human, or non-human primate C3d.

29. The DNA immunization vector according to claim 27, wherein the linear concatamer encodes no more than one wild-type sequence encoding murine, human, or other mammalian C3d.

30. The DNA immunization vector according to claim 27, wherein the linear concatamer encodes an antigen.

31. The DNA immunization vector according to claim 27, wherein the linear concatamer encodes an antigen, and wherein the DNA sequence is non-identical to the naturally occurring DNA sequence.

32. A pharmaceutical composition comprising the DNA immunization vector of claim 27 and a pharmaceutically acceptable excipient.

33. A method of inducing immune response to an antigen in a human or an animal comprising administering a pharmaceutical composition comprising a DNA immunization vector of claim 27 and a pharmaceutically acceptable excipient.

34. A method of introducing a DNA encoding a naturally occurring protein into a human or an animal comprising administering a pharmaceutical composition of claim 32.

35. A method of inducing immune response to an antigen in a human or an animal comprising administering a DNA immunization vector of claim 27.

36. The method according to 35, wherein the administration of DNA results in a therapeutic effect on the human or animal.

37. A method of manufacturing a medicament for inducing immune response to an antigen in a human or an animal using a DNA immunization vector according to claim 27.

38. The DNA immunization vector according to claim 27, wherein the DNA sequence encoding human C3d protein is selected from the group consisting of SEQ ID Nos. 39, 40, 41, 42, 44, 45, 47, 48, and 49.

39. The DNA immunization vector according to claim 27, wherein the DNA sequence encoding murine C3d protein is selected from the group consisting of SEQ ID Nos. 1, 12, 15, 20, 21, 24, 32, and 33.

40. The DNA immunization vector according to claim 27, wherein the DNA sequence encoding non-human primate C3d protein is selected from the group consisting of SEQ ID Nos. 59, 60, and 61.

41. An isolated DNA comprising SEQ ID No. 58, wherein the DNA encodes rhesus macaque C3d protein.


SEQUENCE LISTING (66 sequences)

SEQ ID NO: 1 - DNA, 945 bp, Mus sp.
acccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtggaagccacttcctacgctcttctcgcactgcttctcctgaaggatttcgactccgtgccccctgtagtgcgctggctgaacgaacaacgttactatgggggggggtatggatctacgcaagcaacattcatggtatttcaagccttagctcagtatcagactgatgtaccagaccacaaggatcttaatatggatgtgtccttccacctcccctcatcagggtccggagggggtggatcagggggcggaggttccggtaccagatcctaa

SEQ ID NO: 2 - DNA, 20 bp, Artificial Sequence
  Description of Artificial Sequence Primer
ggtgttccaagctttggccc

SEQ ID NO: 3 - DNA, 20 bp, Artificial Sequence
  Description of Artificial Sequence Primer
gggccaaagcttggaacacc

SEQ ID NO: 4 - DNA, 17 bp, Artificial Sequence
  Description of Artificial Sequence Primer
caggaaacagctatgac

SEQ ID NO: 5 - DNA, 17 bp, Artificial Sequence
  Description of Artificial Sequence Primer
gtaaaacgacggccagt

SEQ ID NO: 6 - DNA, 28 bp, Artificial Sequence
  Description of Artificial Sequence Primer
ggaagctttggcccagtatcagactgat

SEQ ID NO: 7 - DNA, 30 bp, Artificial Sequence
  Description of Artificial Sequence Primer
ggaagctttatttaacgtgtttacgtcgag

SEQ ID NO: 8 - DNA, 52 bp, Artificial Sequence
  Description of Artificial Sequence Primer
gctttggcccagtatcagactgatgtaccagaccacaaggatcttaatatgg

SEQ ID NO: 9 - DNA, 52 bp, Artificial Sequence
  Description of Artificial Sequence Primer
ccatattaagatccttgtggtctggtacatcagtctgatactgggccaaagc

SEQ ID NO: 10 - DNA, 20 bp, Artificial Sequence
  Description of Artificial Sequence Primer
ggttccggtacctgctaacc

SEQ ID NO: 11 - DNA, 20 bp, Artificial Sequence
  Description of Artificial Sequence Primer
ggttagcaggtaccggaacc

SEQ ID NO: 12 - DNA, 914 bp, Mus sp.
accccagcgggctccggagaacaaaacatgattggaatgacgcctacagtcattgcggtccactacctggaccagaccgaacagtgggagaaattcggaatcgagaaacgccaagaagcactggagctgattaaaaagggctatacgcagcagctggccttcaaacaaccttcttcagcttatgctgcctttaataaccgtcctccttctacgtggcttacggcctacgtggtcaaggtattttcactggcagctaacctcattgcgattgatagccacgtgttatgcggcgccgttaaatggttgattctcgagaagcagaagccggatggagtttttcaagaagacggaccggtcattcaccaagagatgattggtggttttcgcaacgccaaggaggcagatgtctcactgacggcattcgtgctcatcgcgcttcaagaagcacgtgacatttgcgaaggacaagtaaacagccttcccggctccattaataaggctggtgagtacattgaggcgtcatatatgaatcttcaacgtccttatacggtcgctatcgcgggctacgccctggccctcatgaacaaacttgaggaaccatacctaggaaaattcctgaatacagccaaggatcgtaatcgttgggaggagcctgatcagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtgttccaagctttggcccaatatcaaacagatgtccctgaccataaggacttgaacatggatgtgtccttccacctccccagcagtggatcctgctagagttctga

SEQ ID NO: 13 - DNA, 25 bp, Artificial Sequence
  Description of Artificial Sequence Primer
cacccgagccggtaccagatccacc

SEQ ID NO: 14 - DNA, 25 bp, Artificial Sequence
  Description of Artificial Sequence Primer
ggtggatctggtaccggctcgggtg

SEQ ID NO: 15 - DNA, 950 bp, Mus sp.
ccagatctacgcctgccggtagtggtgagcaaaatatgatcgggatgacccctactgtgatcgccgtgcactatttagatcaaacggagcaatgggaaaaatttgggattgaaaaacgtcaggaagcgttagaattgattaaaaagggatatacacaacaattagcgtttaagcaaccatcaagcgcgtatgccgcgtttaacaacagaccaccatcaacatggttaaccgcgtatgtcgtgaaagtgtttagtttggcggcgaatttaattgctattgatagtcacgtattatgcggagcggtaaagtggctcatcttagaaaagcaaaaaccagacggcgtgttccaagaagacggaccagtcatccaccaggagatgatcgggggctttagaaatgcgaaagaagcggacgtaagcttaaccgcctttgtattgattgccttacaagaggcgcgcgatatttgcgaaggccaagtgaactctttgccgggatcgattaataaagcgggcgaatacatcgaggcatcctatatgaatttacaacgcccttataccgtagcgatcgccggatacgcgttagcgttaatgaataagttagaagagccatatttggggaaattcttaaatacggcgaaggaccgtaataggtgggaagaaccagatcaacaattgtataatgtcgaagcgaccagttatgcgttgttagcgttattacttttaaaggatttcgatagcgtcccaccagtggtcagatggttaaacgaacagcgctattatggcggaggttacgggagtacacaagcgacgtttatggtctttcaggcgctcgcgcagtaccagacggacgtgccagatcacaaagacctcaatatggacgtcagttttcacttgccatcatccgggagcggcggaggtgggagcggagggggcggtacctccggatcctaa

SEQ ID NO: 16 - DNA, 24 bp, Artificial Sequence
  Description of Artificial Sequence Primer
gtggcttccggaacgccaaggagg

SEQ ID NO: 17 - DNA, 30 bp, Artificial Sequence
  Description of Artificial Sequence Primer
gcaggtaccggatcctccgccccctgatcc

SEQ ID NO: 18 - DNA, 30 bp, Artificial Sequence
  Description of Artificial Sequence Primer
gccggtaccagatctaccccagcgggctcc

SEQ ID NO: 19 - DNA, 22 bp, Artificial Sequence
  Description of Artificial Sequence Primer
gtagcctgggtggagccatagc

SEQ ID NO: 20 - DNA, 950 bp, Mus sp.
ccagatctaccccagcgggctccggagaacaaaacatgattggaatgacgcctacagtcattgcggtccactacctggaccagaccgaacagtgggagaaattcggaatcgagaaacgccaagaagcactggagctgattaaaaagggctatacgcagcagctggccttcaaacaaccttcttcagcttatgctgcctttaataaccgtcctccttctacgtggcttacggcctacgtggtcaaggtattttcactggcagctaacctcattgcgattgatagccacgtgttatgcggcgccgttaaatggttgattctcgagaagcagaagccggatggagtttttcaagaagacggaccggtcattcaccaagagatgattggtggttttcgcaacgccaaggaggcagatgtctcactgacggcattcgtgctcatcgcgcttcaagaagcacgtgacatttgcgaaggacaagtaaacagccttcccggctccattaataaggctggtgagtacattgaggcgtcatatatgaatcttcaacgtccttatacggtcgctatcgcgggctacgccctggccctcatgaacaaacttgaggaaccatacctaggaaaattcctgaatacagccaaggatcgtaatcgttgggaggagcctgatcagcagctctacaacgtggaagccacttcctacgctcttctcgcactgcttctcctgaaggatttcgactccgtgccccctgtagtgcgctggctgaacgaacaacgttactatgggggggggtatggatctacgcaagcaacattcatggtatttcaagccttagctcagtatcagactgatgtaccagaccacaaggatcttaatatggatgtgtccttccacctcccctcatcagggtccggagggggtggatcagggggcggaggatccgtacgcagcttc

SEQ ID NO: 21 - DNA, 2855 bp, Artificial Sequence
  Description of Artificial Sequence Vector pBP68-03
atggccctctggatgcgcctcctgcccctgctggccctgctggccctctgggcgcccgcgcccacccgagccggtaccagatctaccccagcgggctccggagaacaaaacatgattggaatgacgcctacagtcattgcggtccactacctggaccagaccgaacagtgggagaaattcggaatcgagaaacgccaagaagcactggagctgattaaaaagggctatacgcagcagctggccttcaaacaaccttcttcagcttatgctgcctttaataaccgtcctccttctacgtggcttacggcctacgtggtcaaggtattttcactggcagctaacctcattgcgattgatagccacgtgttatgcggcgccgttaaatggttgattctcgagaagcagaagccggatggagtttttcaagaagacggaccggtcattcaccaagagatgattggtggttttcgcaacgccaaggaggcagatgtctcactgacggcattcgtgctcatcgcgcttcaagaagcacgtgacatttgcgaaggacaagtaaacagccttcccggctccattaataaggctggtgagtacattgaggcgtcatatatgaatcttcaacgtccttatacggtcgctatcgcgggctacgccctggccctcatgaacaaacttgaggaaccatacctaggaaaattcctgaatacagccaaggatcgtaatcgttgggaggagcctgatcagcagctctacaacgtggaagccacttcctacgctcttctcgcactgcttctcctgaaggatttcgactccgtgccccctgtagtgcgctggctgaacgaacaacgttactatgggggggggtatggatctacgcaagcaacattcatggtatttcaagccttagctcagtatcagactgatgtaccagaccacaaggatcttaatatggatgtgtccttccacctcccctcatcagggtccggagggggtggatcagggggcggaggatctacgcctgccggtagtggtgagcaaaatatgatcgggatgacccctactgtgatcgccgtgcactatttagatcaaacggagcaatgggaaaaatttgggattgaaaaacgtcaggaagcgttagaattgattaaaaagggatatacacaacaattagcgtttaagcaaccatcaagcgcgtatgccgcgtttaacaacagaccaccatcaacatggttaaccgcgtatgtcgtgaaagtgtttagtttggcggcgaatttaattgctattgatagtcacgtattatgcggagcggtaaagtggctcatcttagaaaagcaaaaaccagacggcgtgttccaagaagacggaccagtcatccaccaggagatgatcgggggctttagaaatgcgaaagaagcggacgtaagcttaaccgcctttgtattgattgccttacaagaggcgcgcgatatttgcgaaggccaagtgaactctttgccgggatcgattaataaagcgggcgaatacatcgaggcatcctatatgaatttacaacgcccttataccgtagcgatcgccggatacgcgttagcgttaatgaataagttagaagagccatatttggggaaattcttaaatacggcgaaggaccgtaataggtgggaagaaccagatcaacaattgtataatgtcgaagcgaccagttatgcgttgttagcgttattacttttaaaggatttcgatagcgtcccaccagtggtcagatggttaaacgaacagcgctattatggcggaggttacgggagtacacaagcgacgtttatggtctttcaggcgctcgcgcagtaccagacggacgtgccagatcacaaagacctcaatatggacgtcagttttcacttgccatcatccgggagcggcggaggtgggagcggagggggcggtacctccggatctacccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagcagtggatccta

SEQ ID NO: 22 - DNA, 93 bp, Homo sapiens
atggatgcaatgaagagagggctctgctgtgtgctgctgctgtgtggagcagtcttcgtttccgctagatctgggtgataaggatcctagtaa

SEQ ID NO: 23 - DNA, 93 bp, Homo sapiens
ttactaggatccttatcacccagatctagcggaaacgaagactgctccacacagcagcagcacacagcagagccctctcttcattgcatccat

SEQ ID NO: 24 - DNA, 5744 bp, Artificial Sequence
  Description of Artificial Sequence Vector pVK68-01
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctgggccgccaccatggatgcaatgaagagagggctctgctgtgtgctgctgctgtgtggagcagtcttcgtttccgctagatctaccccagcgggctccggagaacaaaacatgattggaatgacgcctacagtcattgcggtccactacctggaccagaccgaacagtgggagaaattcggaatcgagaaacgccaagaagcactggagctgattaaaaagggctatacgcagcagctggccttcaaacaaccttcttcagcttatgctgcctttaataaccgtcctccttctacgtggcttacggcctacgtggtcaaggtattttcactggcagctaacctcattgcgattgatagccacgtgttatgcggcgccgttaaatggttgattctcgagaagcagaagccggatggagtttttcaagaagacggaccggtcattcaccaagagatgattggtggttttcgcaacgccaaggaggcagatgtctcactgacggcattcgtgctcatcgcgcttcaagaagcacgtgacatttgcgaaggacaagtaaacagccttcccggctccattaataaggctggtgagtacattgaggcgtcatatatgaatcttcaacgtccttatacggtcgctatcgcgggctacgccctggccctcatgaacaaacttgaggaaccatacctaggaaaattcctgaatacagccaaggatcgtaatcgttgggaggagcctgatcagcagctctacaacgtggaagccacttcctacgctcttctcgcactgcttctcctgaaggatttcgactccgtgccccctgtagtgcgctggctgaacgaacaacgttactatgggggggggtatggatctacgcaagcaacattcatggtatttcaagccttagctcagtatcagactgatgtaccagaccacaaggatcttaatatggatgtgtccttccacctcccctcatcagggtccggagggggtggatcagggggcggaggatctacgcctgccggtagtggtgagcaaaatatgatcgggatgacccctactgtgatcgccgtgcactatttagatcaaacggagcaatgggaaaaatttgggattgaaaaacgtcaggaagcgttagaattgattaaaaagggatatacacaacaattagcgtttaagcaaccatcaagcgcgtatgccgcgtttaacaacagaccaccatcaacatggttaaccgcgtatgtcgtgaaagtgtttagtttggcggcgaatttaattgctattgatagtcacgtattatgcggagcggtaaagtggctcatcttagaaaagcaaaaaccagacggcgtgttccaagaagacggaccagtcatccaccaggagatgatcgggggctttagaaatgcgaaagaagcggacgtaagcttaaccgcctttgtattgattgccttacaagaggcgcgcgatatttgcgaaggccaagtgaactctttgccgggatcgattaataaagcgggcgaatacatcgaggcatcctatatgaatttacaacgcccttataccgtagcgatcgccggatacgcgttagcgttaatgaataagttagaagagccatatttggggaaattcttaaatacggcgaaggaccgtaataggtgggaagaaccagatcaacaattgtataatgtcgaagcgaccagttatgcgttgttagcgttattacttttaaaggatttcgatagcgtcccaccagtggtcagatggttaaacgaacagcgctattatggcggaggttacgggagtacacaagcgacgtttatggtctttcaggcgctcgcgcagtaccagacggacgtgccagatcacaaagacctcaatatggacgtcagttttcacttgccatcatccgggagcggcggaggtgggagcggagggggcggtacctccggatctacccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagcagtggatcctagtaaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 25 - DNA, 80 bp, Artificial Sequence
  Description of Artificial Sequence Synthetic oligonucleotide
agatctcacattgcctctattgctttgaacaacttgaacaagtctggtttggtaggagaaggtgagtctaagaagatttt

SEQ ID NO: 26 - DNA, 80 bp, Artificial Sequence
  Description of Artificial Sequence Synthetic oligonucleotide
gttgaccctaagcatgtttgtgttgagactagagacattcctaagaacgctggatgtttcagagacgacaacggtactga

SEQ ID NO: 27 - DNA, 80 bp, Artificial Sequence
  Description of Artificial Sequence Synthetic oligonucleotide
aacacctgcgttgagaacaacaaccctacttgcgacatcaacaacggtggatgtgacccaaccgcctcttgtcaaaacgc

SEQ ID NO: 28 - DNA, 57 bp, Artificial Sequence
  Description of Artificial Sequence Synthetic oligonucleotide
aaggaaccaacccctaacgcctactacgagggtgttttctgttcttcttccggatcc

SEQ ID NO: 29 - DNA, 80 bp, Artificial Sequence
  Description of Artificial Sequence Synthetic oligonucleotide
gcgttaggggttggttccttacaggtgcaaataatcttcttggagttttcggtagattcagcgttttgacaagaggcggt

SEQ ID NO: 30 - DNA, 80 bp, Artificial Sequence
  Description of Artificial Sequence Synthetic oligonucleotide
ttgttctcaacgcaggtgttaccctcacccttcttgtaacccaacaaacatctccactcttcagtaccgttgtcgtctct

SEQ ID NO: 31 - DNA, 80 bp, Artificial Sequence
  Description of Artificial Sequence Synthetic oligonucleotide
caaacatgcttagggtcaacacccaacaagtccataccgtccatgttcagcatcttagccaaaatcttcttagactcacc

SEQ ID NO: 32 - DNA, 3252 bp, Artificial Sequence
  Description of Artificial Sequence Vector pVK96-01
atggatgcaatgaagagagggctctgctgtgtgctgctgctgtgtggagcagtcttcgtttccgctagatctcacattgcctctattgctttgaacaacttgaacaagtctggtttggtaggagaaggtgagtctaagaagattttggctaagatgctgaacatggacggtatggacttgttgggtgttgaccctaagcatgtttgtgttgacactagagacattcctaagaacgctggatgtttcagagacgacaacggtactgaagagtggagatgtttgttgggttacaagaagggtgagggtaacacctgcgttgagaacaacaaccctacttgcgacatcaacaacggtggatgtgacccaaccgcctcttgtcaaaacgctgaatctaccgaaaactccaagaagattatttgcacctgtaaggaaccaacccctaacgcctactacgagggtgttttctgttcttcttccggatctaccccagcgggctccggagaacaaaacatgattggaatgacgcctacagtcattgcggtccactacctggaccagaccgaacagtgggagaaattcggaatcgagaaacgccaagaagcactggagctgattaaaaagggctatacgcagcagctggccttcaaacaaccttcttcagcttatgctgcctttaataaccgtcctccttctacgtggcttacggcctacgtggtcaaggtattttcactggcagctaacctcattgcgattgatagccacgtgttatgcggcgccgttaaatggttgattctcgagaagcagaagccggatggagtttttcaagaagacggaccggtcattcaccaagagatgattggtggttttcgcaacgccaaggaggcagatgtctcactgacggcattcgtgctcatcgcgcttcaagaagcacgtgacatttgcgaaggacaagtaaacagccttcccggctccattaataaggctggtgagtacattgaggcgtcatatatgaatcttcaacgtccttatacggtcgctatcgcgggctacgccctggccctcatgaacaaacttgaggaaccatacctaggaaaattcctgaatacagccaaggatcgtaatcgttgggaggagcctgatcagcagctctacaacgtggaagccacttcctacgctcttctcgcactgcttctcctgaaggatttcgactccgtgccccctgtagtgcgctggctgaacgaacaacgttactatgggggggggtatggatctacgcaagcaacattcatggtatttcaagccttagctcagtatcagactgatgtaccagaccacaaggatcttaatatggatgtgtccttccacctcccctcatcagggtccggagggggtggatcagggggcggaggatctacgcctgccggtagtggtgagcaaaatatgatcgggatgacccctactgtgatcgccgtgcactatttagatcaaacggagcaatgggaaaaatttgggattgaaaaacgtcaggaagcgttagaattgattaaaaagggatatacacaacaattagcgtttaagcaaccatcaagcgcgtatgccgcgtttaacaacagaccaccatcaacatggttaaccgcgtatgtcgtgaaagtgtttagtttggcggcgaatttaattgctattgatagtcacgtattatgcggagcggtaaagtggctcatcttagaaaagcaaaaaccagacggcgtgttccaagaagacggaccagtcatccaccaggagatgatcgggggctttagaaatgcgaaagaagcggacgtaagcttaaccgcctttgtattgattgccttacaagaggcgcgcgatatttgcgaaggccaagtgaactctttgccgggatcgattaataaagcgggcgaatacatcgaggcatcctatatgaatttacaacgcccttataccgtagcgatcgccggatacgcgttagcgttaatgaataagttagaagagccatatttggggaaattcttaaatacggcgaaggaccgtaataggtgggaagaaccagatcaacaattgtataatgtcgaagcgaccagttatgcgttgttagcgttattacttttaaaggatttcgatagcgtcccaccagtggtcagatggttaaacgaacagcgctattatggcggaggttacgggagtacacaagcgacgtttatggtctttcaggcgctcgcgcagtaccagacggacgtgccagatcacaaagacctcaatatggacgtcagttttcacttgccatcatccgggagcggcggaggtgggagcggagggggcggtacctccggatctacccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagcagtggatcc

SEQ ID NO: 33 - DNA, 3252 bp, Artificial Sequence
  Description of Artificial Sequence Vector pVK96-02
atggatgcaatgaagagagggctctgctgtgtgctgctgctgtgtggagcagtcttcgtttccgctagatctaccccagcgggctccggagaacaaaacatgattggaatgacgcctacagtcattgcggtccactacctggaccagaccgaacagtgggagaaattcggaatcgagaaacgccaagaagcactggagctgattaaaaagggctatacgcagcagctggccttcaaacaaccttcttcagcttatgctgcctttaataaccgtcctccttctacgtggcttacggcctacgtggtcaaggtattttcactggcagctaacctcattgcgattgatagccacgtgttatgcggcgccgttaaatggttgattctcgagaagcagaagccggatggagtttttcaagaagacggaccggtcattcaccaagagatgattggtggttttcgcaacgccaaggaggcagatgtctcactgacggcattcgtgctcatcgcgcttcaagaagcacgtgacatttgcgaaggacaagtaaacagccttcccggctccattaataaggctggtgagtacattgaggcgtcatatatgaatcttcaacgtccttatacggtcgctatcgcgggctacgccctggccctcatgaacaaacttgaggaaccatacctaggaaaattcctgaatacagccaaggatcgtaatcgttgggaggagcctgatcagcagctctacaacgtggaagccacttcctacgctcttctcgcactgcttctcctgaaggatttcgactccgtgccccctgtagtgcgctggctgaacgaacaacgttactatgggggggggtatggatctacgcaagcaacattcatggtatttcaagccttagctcagtatcagactgatgtaccagaccacaaggatcttaatatggatgtgtccttccacctcccctcatcagggtccggagggggtggatcagggggcggaggatctacgcctgccggtagtggtgagcaaaatatgatcgggatgacccctactgtgatcgccgtgcactatttagatcaaacggagcaatgggaaaaatttgggattgaaaaacgtcaggaagcgttagaattgattaaaaagggatatacacaacaattagcgtttaagcaaccatcaagcgcgtatgccgcgtttaacaacagaccaccatcaacatggttaaccgcgtatgtcgtgaaagtgtttagtttggcggcgaatttaattgctattgatagtcacgtattatgcggagcggtaaagtggctcatcttagaaaagcaaaaaccagacggcgtgttccaagaagacggaccagtcatccaccaggagatgatcgggggctttagaaatgcgaaagaagcggacgtaagcttaaccgcctttgtattgattgccttacaagaggcgcgcgatatttgcgaaggccaagtgaactctttgccgggatcgattaataaagcgggcgaatacatcgaggcatcctatatgaatttacaacgcccttataccgtagcgatcgccggatacgcgttagcgttaatgaataagttagaagagccatatttggggaaattcttaaatacggcgaaggaccgtaataggtgggaagaaccagatcaacaattgtataatgtcgaagcgaccagttatgcgttgttagcgttattacttttaaaggatttcgatagcgtcccaccagtggtcagatggttaaacgaacagcgctattatggcggaggttacgggagtacacaagcgacgtttatggtctttcaggcgctcgcgcagtaccagacggacgtgccagatcacaaagacctcaatatggacgtcagttttcacttgccatcatccgggagcggcggaggtgggagcggagggggcggtacctccggatctacccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagcagtggatctcacattgcctctattgctttgaacaacttgaacaagtctggtttggtaggagaaggtgagtctaagaagattttggctaagatgctgaacatggacggtatggacttgttgggtgttgaccctaagcatgtttgtgttgacactagagacattcctaagaacgctggatgtttcagagacgacaacggtactgaagagtggagatgtttgttgggttacaagaagggtgagggtaacacctgcgttgagaacaacaaccctacttgcgacatcaacaacggtggatgtgacccaaccgcctcttgtcaaaacgctgaatctaccgaaaactccaagaagattatttgcacctgtaaggaaccaacccctaacgcctactacgagggtgttttctgttcttcttccggatcc

SEQ ID NO: 34 - DNA, 26 bp, Artificial Sequence
  Description of Artificial Sequence Primer
gggggcagttggagggacacatcaag

SEQ ID NO: 35 - DNA, 24 bp, Artificial Sequence
  Description of Artificial Sequence Primer
ggaccccctcgggctgcggggaac

SEQ ID NO: 36 - DNA, 29 bp, Artificial Sequence
  Description of Artificial Sequence Primer
ggcatatgaccccgtcgggctccggggaa

SEQ ID NO: 37 - DNA, 47 bp, Artificial Sequence
  Description of Artificial Sequence Primer
ggaattcagcaggatccactgctgggcagttggagggacacatcaag

SEQ ID NO: 38 - DNA, 891 bp, Homo sapiens
accccctcgggctccggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatacgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcaactaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgccctga

SEQ ID NO: 39 - DNA, 947 bp, Homo sapiens
ccagatctacgccaagcggatcaggcgagcagaatatgatcgggatgacaccaaccgtaattgcggtccattatctcgacgaaaccgaacagtgggaaaaatttgggctcgaaaagcgtcaaggcgctctcgagttgatcaagaaaggctacacgcaacagttagcgttccgtcaaccatcatcagcgttcgccgctttcgtaaagcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgattggcggcctccgcaataacaatgagaaggacatggcgttaaccgcgtttgtcttaatcagtttacaggaagccaaagacatttgtgaggaacaggtaaatagtttacctgggagtattacgaaagcgggcgatttcttagaagcaaattacatgaatctccaacgctcatacacggtagcgatcgcgggatatgccttagcgcagatggggagattaaaaggcccattactgaacaagtttttaacaaccgcaaaagacaagaataggtgggaggacccaggcaagcaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgcacctgatcaccaagaattaaacttagatgtcagtctgcagttaccaagttcagggtcaggtggaggtggaagtggtggaggtggaagcggatcctaa

SEQ ID NO: 40 - DNA, 947 bp, Homo sapiens
ccagatctactccttcagggagtggagaacaaaacatgattggtatgacccctacagtgatcgccgtacactacttagatgagacagagcaatgggagaaattcggtttggagaaaagacagggagcgttagaacttattaaaaagggatatacacagcaactcgcttttaggcagcctagtagcgcatttgctgcgtttgtcaaaagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgatcgggggcttgagaaacaataacgaaaaagatatggctctgacagctttcgtgctgatttccctccaagaggcgaaggatatctgcgaagagcaagtgaactcactcccaggatcaatcaccaaggccggggactttctggaggcgaactatatgaacttgcagaggagctataccgtcgcaattgccggttacgcgctcgcacaaatgggacgtctcaaaggacctctgttaaataaattcctcacgacggcgaaggataaaaaccgatgggaagaccctgggaaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgcgccagaccaccaggagcttaatctcgacgtatcattacaactcccttcaagcggcagcggcgggggcgggtcaggaggcgggggttctggatcctaa

SEQ ID NO: 41 - DNA, 2897 bp, Artificial Sequence
  Description of Artificial Sequence Vector pBP80-01
atggccctctggatgcgcctcctgcccctgctggccctgctggccctctgggcgcccgcgcccacccgagccggatccagatctacgccaagcggatcaggcgagcagaatatgatcgggatgacaccaaccgtaattgcggtccattatctcgacgaaaccgaacagtgggaaaaatttgggctcgaaaagcgtcaaggcgctctcgagttgatcaagaaaggctacacgcaacagttagcgttccgtcaaccatcatcagcgttcgccgctttcgtaaagcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgattggcggcctccgcaataacaatgagaaggacatggcgttaaccgcgtttgtcttaatcagtttacaggaagccaaagacatttgtgaggaacaggtaaatagtttacctgggagtattacgaaagcgggcgatttcttagaagcaaattacatgaatctccaacgctcatacacggtagcgatcgcgggatatgccttagcgcagatggggagattaaaaggcccattactgaacaagtttttaacaaccgcaaaagacaagaataggtgggaggacccaggcaagcaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgcacctgatcaccaagaattaaacttagatgtcagtctgcagttaccaagttcagggtcaggtggaggtggaagtggtggaggtggaagcggatctactccttcagggagtggagaacaaaacatgattggtatgacccctacagtgatcgccgtacactacttagatgagacagagcaatgggagaaattcggtttggagaaaagacagggagcgttagaacttattaaaaagggatatacacagcaactcgcttttaggcagcctagtagcgcatttgctgcgtttgtcaaaagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgatcgggggcttgagaaacaataacgaaaaagatatggctctgacagctttcgtgctgatttccctccaagaggcgaaggatatctgcgaagagcaagtgaactcactcccaggatcaatcaccaaggccggggactttctggaggcgaactatatgaacttgcagaggagctataccgtcgcaattgccggttacgcgctcgcacaaatgggacgtctcaaaggacctctgttaaataaattcctcacgacggcgaaggataaaaaccgatgggaagaccctgggaaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgcgccagaccaccaggagcttaatctcgacgtatcattacaactcccttcaagcggcagcggcgggggcgggtcaggaggcgggggttctggatctaccccctcgggctccggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagcagtggatcctgctgactcgaggcctgcagggcggccgcttaattaattga

SEQ ID NO: 42 - DNA, 2844 bp, Artificial Sequence
  Description of Artificial Sequence Vector pVK80-01
atggatgcaatgaagagagggctctgctgtgtgctgctgctgtgtggagcagtcttcgtttccgctagatctacgccaagcggatcaggcgagcagaatatgatcgggatgacaccaaccgtaattgcggtccattatctcgacgaaaccgaacagtgggaaaaatttgggctcgaaaagcgtcaaggcgctctcgagttgatcaagaaaggctacacgcaacagttagcgttccgtcaaccatcatcagcgttcgccgctttcgtaaagcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgattggcggcctccgcaataacaatgagaaggacatggcgttaaccgcgtttgtcttaatcagtttacaggaagccaaagacatttgtgaggaacaggtaaatagtttacctgggagtattacgaaagcgggcgatttcttagaagcaaattacatgaatctccaacgctcatacacggtagcgatcgcgggatatgccttagcgcagatggggagattaaaaggcccattactgaacaagtttttaacaaccgcaaaagacaagaataggtgggaggacccaggcaagcaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgcacctgatcaccaagaattaaacttagatgtcagtctgcagttaccaagttcagggtcaggtggaggtggaagtggtggaggtggaagcggatctactccttcagggagtggagaacaaaacatgattggtatgacccctacagtgatcgccgtacactacttagatgagacagagcaatgggagaaattcggtttggagaaaagacagggagcgttagaacttattaaaaagggatatacacagcaactcgcttttaggcagcctagtagcgcatttgctgcgtttgtcaaaagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgatcgggggcttgagaaacaataacgaaaaagatatggctctgacagctttcgtgctgatttccctccaagaggcgaaggatatctgcgaagagcaagtgaactcactcccaggatcaatcaccaaggccggggactttctggaggcgaactatatgaacttgcagaggagctataccgtcgcaattgccggttacgcgctcgcacaaatgggacgtctcaaaggacctctgttaaataaattcctcacgacggcgaaggataaaaaccgatgggaagaccctgggaaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgcgccagaccaccaggagcttaatctcgacgtatcattacaactcccttcaagcggcagcggcgggggcgggtcaggaggcgggggttctggatctaccccctcgggctccggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagcagtggatcc

SEQ ID NO: 43 - DNA, 309 bp, Plasmodium falciparum
agatctaacattgcccaacaccaatgcgttaagaagcaatgtccacaaaactccggatgtttcagacatctggacgagagagaagaatgtaagtgtctgttgaactacaagcaggaaggtgataagtgtgttgagaacccaaaccctacctgtaacgagaacaacggtggatgcgacgctgacgctaagtgcaccgaagaagactctggttctaacggaaagaagattacttgcgaatgtactaagccagactcttaccctttgttcgatggaatcttctgttcttcctctaactcttcctctggatcc

SEQ ID NO: 44 - DNA, 3147 bp, Artificial Sequence
  Description of Artificial Sequence Vector pVK104-01
atggatgcaatgaagagagggctctgctgtgtgctgctgctgtgtggagcagtcttcgtttccgctagatctaacattgcccaacaccaatgcgttaagaagcaatgtccacaaaactccggatgtttcagacatctggacgagagagaagaatgtaagtgtctgttgaactacaagcaggaaggtgataagtgtgttgagaacccaaaccctacctgtaacgagaacaacggtggatgcgacgctgacgctaagtgcaccgaagaagactctggttctaacggaaagaagattacttgcgaatgtactaagccagactcttaccctttgttcgatggaatcttctgttcttcctctaactcttcctctggatctacgccaagcggatcaggcgagcagaatatgatcgggatgacaccaaccgtaattgcggtccattatctcgacgaaaccgaacagtgggaaaaatttgggctcgaaaagcgtcaaggcgctctcgagttgatcaagaaaggctacacgcaacagttagcgttccgtcaaccatcatcagcgttcgccgctttcgtaaagcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgattggcggcctccgcaataacaatgagaaggacatggcgttaaccgcgtttgtcttaatcagtttacaggaagccaaagacatttgtgaggaacaggtaaatagtttacctgggagtattacgaaagcgggcgatttcttagaagcaaattacatgaatctccaacgctcatacacggtagcgatcgcgggatatgccttagcgcagatggggagattaaaaggcccattactgaacaagtttttaacaaccgcaaaagacaagaataggtgggaggacccaggcaagcaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgcacctgatcaccaagaattaaacttagatgtcagtctgcagttaccaagttcagggtcaggtggaggtggaagtggtggaggtggaagcggatctactccttcagggagtggagaacaaaacatgattggtatgacccctacagtgatcgccgtacactacttagatgagacagagcaatgggagaaattcggtttggagaaaagacagggagcgttagaacttattaaaaagggatatacacagcaactcgcttttaggcagcctagtagcgcatttgctgcgtttgtcaaaagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgatcgggggcttgagaaacaataacgaaaaagatatggctctgacagctttcgtgctgatttccctccaagaggcgaaggatatctgcgaagagcaagtgaactcactcccaggatcaatcaccaaggccggggactttctggaggcgaactatatgaacttgcagaggagctataccgtcgcaattgccggttacgcgctcgcacaaatgggacgtctcaaaggacctctgttaaataaattcctcacgacggcgaaggataaaaaccgatgggaagaccctgggaaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgcgccagaccaccaggagcttaatctcgacgtatcattacaactcccttcaagcggcagcggcgggggcgggtcaggaggcgggggttctggatctaccccctcgggctccggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagcagtggatcc

SEQ ID NO: 45 - DNA, 3147 bp, Artificial Sequence
  Description of Artificial Sequence Vector pVK104-02
atggatgcaatgaagagagggctctgctgtgtgctgctgctgtgtggagcagtcttcgtttccgctagatctacgccaagcggatcaggcgagcagaatatgatcgggatgacaccaaccgtaattgcggtccattatctcgacgaaaccgaacagtgggaaaaatttgggctcgaaaagcgtcaaggcgctctcgagttgatcaagaaaggctacacgcaacagttagcgttccgtcaaccatcatcagcgttcgccgctttcgtaaagcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgattggcggcctccgcaataacaatgagaaggacatggcgttaaccgcgtttgtcttaatcagtttacaggaagccaaagacatttgtgaggaacaggtaaatagtttacctgggagtattacgaaagcgggcgatttcttagaagcaaattacatgaatctccaacgctcatacacggtagcgatcgcgggatatgccttagcgcagatggggagattaaaaggcccattactgaacaagtttttaacaaccgcaaaagacaagaataggtgggaggacccaggcaagcaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgcacctgatcaccaagaattaaacttagatgtcagtctgcagttaccaagttcagggtcaggtggaggtggaagtggtggaggtggaagcggatctactccttcagggagtggagaacaaaacatgattggtatgacccctacagtgatcgccgtacactacttagatgagacagagcaatgggagaaattcggtttggagaaaagacagggagcgttagaacttattaaaaagggatatacacagcaactcgcttttaggcagcctagtagcgcatttgctgcgtttgtcaaaagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgatcgggggcttgagaaacaataacgaaaaagatatggctctgacagctttcgtgctgatttccctccaagaggcgaaggatatctgcgaagagcaagtgaactcactcccaggatcaatcaccaaggccggggactttctggaggcgaactatatgaacttgcagaggagctataccgtcgcaattgccggttacgcgctcgcacaaatgggacgtctcaaaggacctctgttaaataaattcctcacgacggcgaaggataaaaaccgatgggaagaccctgggaaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgcgccagaccaccaggagcttaatctcgacgtatcattacaactcccttcaagcggcagcggcgggggcgggtcaggaggcgggggttctggatctaccccctcgggctccggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagcagtggatctaacattgcccaacaccaatgcgttaagaagcaatgtccacaaaactccggatgtttcagacatctggacgagagagaagaatgtaagtgtctgttgaactacaagcaggaaggtgataagtgtgttgagaacccaaaccctacctgtaacgagaacaacggtggatgcgacgctgacgctaagtgcaccgaagaagactctggttctaacggaaagaagattacttgcgaatgtactaagccagactcttaccctttgttcgatggaatcttctgttcttcctctaactcttcctctggatcc

SEQ ID NO: 46 - DNA, 309 bp, Plasmodium falciparum
agatctaacattgcccaacaccaatgcgttaagaagcaaattccacaaaactccggatgtttcagacatctggacgagagagaagaatggaagtgtctgttgaactacaagcaggaaggtgataagtgtgttgagaacccaaaccctacctgtaacgagaacaacggtggatgcgacgctgacgctaagtgcaccgaagaagactctggttctaacggaaagaagattacttgcgaatgtactaagccagactcttaccctttgttcgatggaatcttctgttcttcctctaactcttcctctggatcc

SEQ ID NO: 47 - DNA, 3147 bp, Artificial Sequence
  Description of Artificial Sequence Vector pVK104-03
atggatgcaatgaagagagggctctgctgtgtgctgctgctgtgtggagcagtcttcgtttccgctagatctaacattgcccaacaccaatgcgttaagaagcaaattccacaaaactccggatgtttcagacatctggacgagagagaagaatggaagtgtctgttgaactacaagcaggaaggtgataagtgtgttgagaacccaaaccctacctgtaacgagaacaacggtggatgcgacgctgacgctaagtgcaccgaagaagactctggttctaacggaaagaagattacttgcgaatgtactaagccagactcttaccctttgttcgatggaatcttctgttcttcctctaactcttcctctggatctacgccaagcggatcaggcgagcagaatatgatcgggatgacaccaaccgtaattgcggtccattatctcgacgaaaccgaacagtgggaaaaatttgggctcgaaaagcgtcaaggcgctctcgagttgatcaagaaaggctacacgcaacagttagcgttccgtcaaccatcatcagcgttcgccgctttcgtaaagcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgattggcggcctccgcaataacaatgagaaggacatggcgttaaccgcgtttgtcttaatcagtttacaggaagccaaagacatttgtgaggaacaggtaaatagtttacctgggagtattacgaaagcgggcgatttcttagaagcaaattacatgaatctccaacgctcatacacggtagcgatcgcgggatatgccttagcgcagatggggagattaaaaggcccattactgaacaagtttttaacaaccgcaaaagacaagaataggtgggaggacccaggcaagcaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgcacctgatcaccaagaattaaacttagatgtcagtctgcagttaccaagttcagggtcaggtggaggtggaagtggtggaggtggaagcggatctactccttcagggagtggagaacaaaacatgattggtatgacccctacagtgatcgccgtacactacttagatgagacagagcaatgggagaaattcggtttggagaaaagacagggagcgttagaacttattaaaaagggatatacacagcaactcgcttttaggcagcctagtagcgcatttgctgcgtttgtcaaaagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgatcgggggcttgagaaacaataacgaaaaagatatggctctgacagctttcgtgctgatttccctccaagaggcgaaggatatctgcgaagagcaagtgaactcactcccaggatcaatcaccaaggccggggactttctggaggcgaactatatgaacttgcagaggagctataccgtcgcaattgccggttacgcgctcgcacaaatgggacgtctcaaaggacctctgttaaataaattcctcacgacggcgaaggataaaaaccgatgggaagaccctgggaaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgcgccagaccaccaggagcttaatctcgacgtatcattacaactcccttcaagcggcagcggcgggggcgggtcaggaggcgggggttctggatctaccccctcgggctccggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagcagtggatcc

SEQ ID NO: 48 - DNA, 3147 bp, Artificial Sequence
  Description of Artificial Sequence Vector pVK104-04
atggatgcaatgaagagagggctctgctgtgtgctgctgctgtgtggagcagtcttcgtttccgctagatctacgccaagcggatcaggcgagcagaatatgatcgggatgacaccaaccgtaattgcggtccattatctcgacgaaaccgaacagtgggaaaaatttgggctcgaaaagcgtcaaggcgctctcgagttgatcaagaaaggctacacgcaacagttagcgttccgtcaaccatcatcagcgttcgccgctttcgtaaagcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgattggcggcctccgcaataacaatgagaaggacatggcgttaaccgcgtttgtcttaatcagtttacaggaagccaaagacatttgtgaggaacaggtaaatagtttacctgggagtattacgaaagcgggcgatttcttagaagcaaattacatgaatctccaacgctcatacacggtagcgatcgcgggatatgccttagcgcagatggggagattaaaaggcccattactgaacaagtttttaacaaccgcaaaagacaagaataggtgggaggacccaggcaagcaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgcacctgatcaccaagaattaaacttagatgtcagtctgcagttaccaagttcagggtcaggtggaggtggaagtggtggaggtggaagcggatctactccttcagggagtggagaacaaaacatgattggtatgacccctacagtgatcgccgtacactacttagatgagacagagcaatgggagaaattcggtttggagaaaagacagggagcgttagaacttattaaaaagggatatacacagcaactcgcttttaggcagcctagtagcgcatttgctgcgtttgtcaaaagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgatcgggggcttgagaaacaataacgaaaaagatatggctctgacagctttcgtgctgatttccctccaagaggcgaaggatatctgcgaagagcaagtgaactcactcccaggatcaatcaccaaggccggggactttctggaggcgaactatatgaacttgcagaggagctataccgtcgcaattgccggttacgcgctcgcacaaatgggacgtctcaaaggacctctgttaaataaattcctcacgacggcgaaggataaaaaccgatgggaagaccctgggaaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgcgccagaccaccaggagcttaatctcgacgtatcattacaactcccttcaagcggcagcggcgggggcgggtcaggaggcgggggttctggatctaccccctcgggctccggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagcagtggatctaacattgcccaacaccaatgcgttaagaagcaaattccacaaaactccggatgtttcagacatctggacgagagagaagaatggaagtgtctgttgaactacaagcaggaaggtgataagtgtgttgagaacccaaaccctacctgtaacgagaacaacggtggatgcgacgctgacgctaagtgcaccgaagaagactctggttctaacggaaagaagattacttgcgaatgtactaagccagactcttaccctttgttcgatggaatcttctgttcttcctctaactcttcctctggatcc

SEQ ID NO: 49 - DNA, 888 bp, Homo sapiens
acaccgtctggtagcggtgagcaaaatatgataggaatgactccgactgttatagcagttcactatttagacgagactgaacaatgggaaaagtttggactggaaaaaaggcaaggtgcactggaattaataaaaaaaggttatacgcagcaactagcgttcaggcagccgtccagcgctttcgcagcatttgtcaagagggctccgtccacttggttgacggcatatgtcgtgaaagtttttagtttggcagttaacttgatagcgatcgatagccaggttttgtgtggtgcagtaaagtggttgatactcgaaaagcaaaagccggatggtgtttttcaagaagacgccccggttatccatcaggagatgatcggaggtctgaggaataataatgaaaaggatatggcattgactgcattcgtattgataagcttgcaagaagcaaaggacatatgtgaagaacaagttaattccttgccgggttccataacaaaggctggtgattttctcgaggctaattatatgaatctgcaacgaagttatacagttgctatagcagggtacgcactcgctcaaatgggtcgcttgaagggtccgctcctgaataagttcttgactactgctaaggacaaaaatagatgggaagatccgggaaaacaactgtataatgttgaagctactagctacgctttgctggctctgttgcaactgaaggatttcgatttcgttcccccggttgttaggtggttaaacgagcaacgctattatggcggaggttacgggtcgactcaagctacatttatggtttttcaggctctggcccagtatcagaaagatgctcccgatcatcaagagctcaatctggacgttagcttgcagttgccg

SEQ ID NO: 50 - DNA, 27 bp, Artificial Sequence
  Description of Artificial Sequence Primer
tgyggrgarcagaacatgatyggcatg

SEQ ID NO: 51 - DNA, 26 bp, Artificial Sequence
  Description of Artificial Sequence Primer
ccgtagtatctyasntcrttgagcca

SEQ ID NO: 52 - DNA, 23 bp, Artificial Sequence
  Description of Artificial Sequence Primer
ggagtcttcgaggagaatgggcc

SEQ ID NO: 53 - DNA, 28 bp, Artificial Sequence
  Description of Artificial Sequence Primer
gtgtgtcwggrrcraagccrgtcatcat

SEQ ID NO: 54 - DNA, 27 bp, Artificial Sequence
  Description of Artificial Sequence Primer
gtratgcaggacttcttcatygacctg

SEQ ID NO: 55 - DNA, 24 bp, Artificial Sequence
  Description of Artificial Sequence Primer
ggctgtcagggacacgtctttctc

SEQ ID NO: 56 - DNA, 25 bp, Artificial Sequence
  Description of Artificial Sequence Primer
gcaagggaccccmgtggcccagatg

SEQ ID NO: 57 - DNA, 23 bp, Artificial Sequence
  Description of Artificial Sequence Primer
gycaccaccgacaakgtgccttg

SEQ ID NO: 58 - DNA, 888 bp, Macaca mulatta
accccctcgggctgcggagaacagaacatgatcaccatgacgcccacagtcatcgctgtgcattacctggatgaaacggaacagtgggagaagttcggcccggagaagcggcagggggccttggagctcatcaagaaggggtacacccagcagctggccttcagacaacccagctctgcctttgcggccttcctgaaccgggcacccagcacctggctgaccgcctacgtggtcaaggtcttctctctggctgtcaacctcattgccatcgactcccaggtcctctgcggggctgttaaatggctgatcctggagaagcagaagcccgacggggtcttccaggaggatgcgcccgtgatacatcaagaaatgactggtggattccggaacaccaacgagaaagacatggccctcacggcctttgttctcatctcgctgcaagaggctaaagagatttgcgaggagcaggtcaacagcctgcccggcagcatcactaaagcaggagacttccttgaagccaactacatgaacctacagagatcctacactgtggccatcgctgcctatgccctggcccagatgggcaggctgaagggacctcttctcaacaaatttctgaccacagccaaagataagaaccgctgggaggagcctggtcagcagctctacaatgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggatgtccctgatcacaaggaactgaacctggatgtgtccctccaactgccc

SEQ ID NO: 59 - DNA, 888 bp, Macaca mulatta
acgccaagcggatcaggcgagcagaatatgatcactatgacaccaaccgtaattgcggtccattatctcgacgaaaccgaacagtgggaaaaatttgggccggaaaagcgtcaaggcgctctcgagttgatcaagaaaggctacacgcaacagttagcgttccgtcaaccatcatcagcgttcgccgctttcctgaatcgtgcgccatcaacgtggctcacagcgtatgtagtgaaggtatttagcctcgccgtaaatttaatcgcgattgacagtcaagtgttatgcggcgcggtcaagtggctcattcttgaaaagcaaaagccagatggcgtattccaagaggacgccccagtcatccaccaagagatgacaggcggctttcgcaatactaatgagaaggacatggcgttaaccgcgtttgtcttaatcagtttacaggaagccaaagaaatttgtgaggaacaggtaaatagtttacctgggagtattacgaaagcgggcgatttcttagaagcaaattacatgaatctccaacgctcatacacggtagcgatcgcggcttatgccttagcgcagatggggagattaaaaggcccattactgaacaagtttttaacaaccgcaaaagacaagaataggtgggaggaaccaggccaacaactttataacgtcgaagcaacgtcatacgcattattagcactcttacaactcaaggacttcgacttcgtaccacctgtggtacggtggcttaacgaacaaaggtattacgggggcggatacggcagcacgcaagcgactttcatggtctttcaagcactcgcacagtaccagaaggatgttcctgatcacaaggaattaaacttagatgtcagtctgcagttacca

SEQ ID NO: 60 - DNA, 888 bp, Macaca mulatta
actccttcagggagtggagaacaaaacatgattacaatgacccctacagtgatcgccgtacactacttagatgagacagagcaatgggagaaattcggtcccgagaaaagacagggagcgttagaacttattaaaaagggatatacacagcaactcgcttttaggcagcctagtagcgcatttgctgcgtttctcaacagagcccctagtacatggttaacggcttacgtcgtaaaagtgttctcattagcggtgaacctgattgcaatcgattcgcaggtactgtgtggagccgtgaaatggttaatcttagagaaacagaaacctgacggagtgtttcaggaagatgcacctgtaattcaccaggaaatgaccgggggcttcagaaacacaaacgaaaaagatatggctctgacagctttcgtgctgatttccctccaagaggcgaaggagatctgcgaagagcaagtgaactcactcccaggatcaatcaccaaggccggggactttctggaggcgaactatatgaacttgcagaggagctataccgtcgcaattgccgcatacgcgctcgcacaaatgggacgtctcaaaggacctctgttaaataaattcctcacgacggcgaaggataaaaaccgatgggaagaacctgggcaacagttgtacaatgtagaggcgaccagttatgcgctgctcgcgttgctccagttgaaagattttgattttgtccctccagtagtcagatggttgaatgagcagcgttactatggaggggggtatggatcaacacaggcaacgtttatggtattccaggcgttagcgcaatatcaaaaagacgtgccagaccacaaagagcttaatctcgacgtatcattacaactccct

SEQ ID NO: 61 - DNA, 888 bp, Macaca mulatta
acaccgtctggtagcggtgagcaaaatatgataaccatgactccgactgttatagcagttcactatttagacgagactgaacaatgggaaaagtttggaccggaaaaaaggcaaggtgcactggaattaataaaaaaaggttatacgcagcaactagcgttcaggcagccgtccagcgctttcgcagcatttctgaacagggctccgtccacttggttgacggcatatgtcgtgaaagtttttagtttggcagttaacttgatagcgatcgatagccaggttttgtgtggtgcagtaaagtggttgatactcgaaaagcaaaagccggatggtgtttttcaagaagacgccccggttatccatcaggagatgactggaggtttcaggaataccaatgaaaaggatatggcattgactgcattcgtattgataagcttgcaagaagcaaaggagatatgtgaagaacaagttaattccttgccgggttccataacaaaggctggtgattttctcgaggctaattatatgaatctgcaacgaagttatacagttgctatagcagcctacgcactcgctcaaatgggtcgcttgaagggtccgctcctgaataagttcttgactactgctaaggacaaaaatagatgggaagagccgggacagcaactgtataatgttgaagctactagctacgctttgctggctctgttgcaactgaaggatttcgatttcgttcccccggttgttaggtggttaaacgagcaacgctattatggcggaggttacgggtcgactcaagctacatttatggtttttcaggctctggcccagtatcagaaagatgtccccgatcataaggagctcaatctggacgttagcttgcagttgccg

SEQ ID NO: 62 - protein, 10 aa, Artificial Sequence
  Description of Artificial Sequence Linker peptide
SGGGGSGGGG

SEQ ID NO: 63 - DNA, 10 bp, Artificial Sequence
  Description of Artificial Sequence Illustrative Kozak sequence
gccaccatgg

SEQ ID NO: 64 - protein, 5 aa, Artificial Sequence
  Description of Artificial Sequence Synthetic peptide
SSGSC

SEQ ID NO: 65 - DNA, 18 bp, Artificial Sequence
  Description of Artificial Sequence Synthetic oligonucleotide
tcagcaggatccactgct

SEQ ID NO: 66 - protein, 5 aa, Artificial Sequence
  Description of Artificial Sequence Synthetic peptide
GGGSG